(19) United States
(12) Patent Application Publication
(10) Pub. No.: US 2016/0331357 A1
(43) Pub. Date: Nov. 17, 2016

(54) SAMPLE COLLECTION DEVICES AND ASSOCIATED SYSTEMS AND METHODS

(71) Applicants: Nanocytomics, LLC, Evanston, IL (US); Northwestern University, Evanston, IL (US)

(72) Inventors: Jarema S. Czarnecki, Evanston, IL (US); Hariharan Subramanian, Evanston, IL (US); Yangrong (Elaine) Zhang, Evanston, IL (US); Vadim Backman, Evanston, IL (US); Hemant Roy, Evanston, IL (US)

(21) Appl. No.: 15/156,056

(22) Filed: May 16, 2016

Related U.S. Application Data
(60) Provisional application No. 62/162,309, filed on May 15, 2015.

(51) Int. Cl. A61B 10/02; A61B 10/04; A61B 42/10

(52) U.S. Cl. CPC A61B 10/02; A61B 42/10; A61B 10/04; A61B 10/0291; A61B 2010/0216; A61B 2017/00473

(57) ABSTRACT

A sample collection apparatus or device, various abrasive devices or components, and methods for their use, are provided for collecting samples from within a mammalian body. Tissue collection and sample preparation is the gold standard in cytology. Many cell scrapers and abrasives and their respective methods of collection destroy cellular tissue, decreasing the quality of the sample. Devices for obtaining cell and tissue samples, can comprise a collection device which is inserted into a cavity, a finger worn collection device, a glove based collection device and respective abrasive instruments or components.

### PRIORITY CLAIM

This application claims priority to U.S. Provisional Patent Application Ser. No. 62/162,309 which was filed on May 15, 2015, the entire contents of which are incorporated herein by reference and relied upon.

### STATEMENT REGARDING FEDERAL FUNDING

This invention was made with government support under R44CA168185, R42CA168055, U01CA111257, and R01CA165309 awarded by the National Institutes of Health. The government has certain rights in the invention.

### FIELD

The present technology relates generally to sample collection devices, and more specifically, to cytology devices for the collection of mammalian tissue, and associated systems and methods.

### BACKGROUND

Existing cytology techniques such as Pap Smear tests have been the state of art for decades. The development of minimally invasive methods and devices in recent years has revolutionized the practice of medicine. The ability to quickly obtain high quality samples with little discomfort has generally made such procedures more acceptable. However, obtaining proper samples for diagnostic testing can be a challenge because certain collection areas are difficult to access and may require more intensive collection and biopsy procedures.

Certain sample collection devices in the market may have limitations or drawbacks that prevent adequate sample collection. For example, some devices lack proper protection against contaminates. Additionally, certain devices are complex and difficult to navigate, which can lead to even more poor tissue collection. Further, devices with limited or no tactile feedback often prevent sufficient or adequate collection. Moreover, the length of many devices is not ideal and also leads to unsatisfactory collection. Accessing the proper collection site is also more difficult with some devices since they are too long, rigid, inconsistent, or difficult to control. Some devices may even cause added discomfort to a patient from contact between the device and tissue of the patient. Several collection methods require that a specific area is sampled making the configuration or design of the device a critical feature. Therefore, there remains a need for improved collection devices that can provide high quality samples.

### SUMMARY

Methods and materials are provided for the collection of biological samples from a patient.

The present disclosure provides sample collection devices comprising an outer shield assembly comprising a distal end, a proximal end and a lumen extending therebetween, wherein the distal end comprises a flexible tip portion; and an inner component comprising a distal end, a proximal end and a lumen extending therebetween, wherein the distal end of the inner component is configured to be inserted into the lumen of the outer shield assembly at its proximal end and to protrude past the distal end of the outer shield assembly thereby penetrating the flexible tip portion when the outer shield assembly and the inner component are in an assembled configuration. In one embodiment, the sample collection device further comprises a sample component having an abrasive head portion at a distal end, wherein the sample component is configured to be inserted into the lumen of the inner component at its proximal end and to protrude past the distal ends of the inner component and outer shield assembly when the outer shield assembly and the inner component are in an assembled configuration. In another embodiment, the sample collection device further comprises at least one of a vacuum tube, an irrigation conduit, an optical sensor, or a camera. In one embodiment, the inner component comprises a stopper secured or attached to the proximal end for contacting the proximal end of the outer shield assembly and thereby limiting a distance the distal end of the inner component can protrude past the distal end of the outer shield assembly when the outer shield assembly and the inner component are in an assembled configuration. In one embodiment, the flexible tip portion is configured to open when the distal end of the inner component protrudes past the distal end of the outer shield assembly. In another embodiment, the flexible tip portion is unitarily or monolithically formed with the outer shield assembly. In yet another embodiment, the flexible tip portion is rounded and outwardly spreadable relative to the distal end of the outer shield assembly. In one embodiment, the sample collection device further comprises a handle comprising a distal end and proximal end coupled the outer shield assembly. In one embodiment, the sample collection device further comprises a protective barrier secured or coupled to the distal end of the handle. In one embodiment, the abrasive head portion comprises one or more bristles. In another embodiment, the sample component is configured to be inserted into a cavity of a patient to a desired sample collection site in the patient through the lumen of the inner component. In another embodiment, the sample component comprises a threaded portion at a proximal end. In yet another embodiment, the threaded portion of the sample component is configured to securably attach to a threaded fitting. In still another embodiment, when the threaded portion of the sample component is securably attached to a threaded fitting, rotational force applied to the threaded fitting imparts a rotational motion on the sample component. In still another embodiment, the inner component is configured to be releasably secured to the outer shield assembly.

In another aspect, the present disclosure provides sample collection devices comprising an outer shell configured to conform to a finger of a human subject, the outer shell comprising a proximal end and a distal end, wherein the proximal end comprises an opening to receive said finger; and a channel on an interior dorsal surface of the device having a first channel opening in proximity to the proximal end of the device and a second channel opening in proximity to the distal end of the device. In one embodiment, the channel shares a wall or surface with an interior portion of the shell. In one embodiment, the channel is configured to receive a sample component, pretreatment component, or a vacuum tube. In one embodiment, the outer shell is configured to include a molded groove in a bottom or lower portion of the distal end of the outer shell. In another embodiment, the distal end of the outer shell is covered by a moveable flap or cover. In yet another embodiment, the sample collection device further comprises a tab on a top or upper portion of the proximal end of the shell.

In another aspect, the present disclosure provides glove-based sample collection devices configured to be worn on at least a finger portion of a hand of a human subject comprising an inner layer that is protected by at least one outer layer; and wherein the inner layer comprises a sample component positioned in proximity to a distal tip of a finger portion of the inner layer, and wherein the sample component is configured for collecting a sample. In one embodiment, the sample component comprises at least one of plastic, silicone, multiple bristles, abrasive, foam, or an adhesive. In one embodiment, the sample component is configured to be expandable. In one embodiment, sample component is configured to be protected and/or covered by the outer layer during insertion and/or retraction of the collection device into and out of a patient cavity. In one embodiment, the sample component is attached to a string. In another embodiment, the glove-based sample collection device further comprises a stopper component positioned on the outer and/or inner layer, wherein the stopper is configured to prevent the sample component from being inserted farther into a patient cavity. In yet another embodiment, a distal tip of the at least one outer layer is configured to open or release once the sample component is inserted.

In another aspect, the present disclosure provides glove-based sample collection devices configured to be worn on at least a finger portion of a hand of a human subject comprising an inner layer that is protected by at least one outer layer; and a channel on an interior dorsal surface of the outer layer having a first channel opening in proximity to the proximal end of the glove-based sample collection device, a second channel opening in proximity to the distal end of the glove-based sample collection device, and a lumen extending therebetween. In one embodiment, the second channel opening comprises a protective covering. In one embodiment, the channel is configured to receive a sample component, pretreatment component, or a vacuum tube. In another embodiment, the channel is unitarily or monolithically formed with the outer layer. In yet another embodiment, the sample component is configured to be protected and/or covered by the channel during insertion and/or retraction of the collection device into and out of a patient cavity.

In another aspect, the present disclosure provides sample collection devices comprising a flexible sample component configured to conform to a finger of a human subject, the flexible sample component comprising a distal end, a proximal end and a lumen therebetween, wherein an inside surface of the distal end and the lumen are configured to receive said finger; an abrasive head portion on an outside surface of the distal end of the sample component; and a removal component securably attached to the inside surface of the distal end of the flexible sample component. In another embodiment, the sample collection device further comprises a positioning component coupled to the proximal end of the flexible sample component.

In another aspect, the present disclosure provides sample collection devices comprising an outer shield assembly comprising a distal end, a proximal end and a lumen extending therebetween, wherein the distal end comprises a flexible tip portion; an inner component comprising a distal end, a proximal end and a lumen extending therebetween, wherein the proximal end comprises an opening to receive a finger of a human subject, and further wherein the distal end comprises an abrasive head; and an adaptor configured to be inserted into the lumen of the outer shield assembly at its proximal end and guide the inner component to protrude past the distal end of the outer shield assembly thereby penetrating the flexible tip portion. In one embodiment, the inner component is configured to be releasably secured to the adaptor.

In another aspect, the present disclosure provides sample collection devices comprising an outer shield assembly comprising a distal end, a proximal end and a lumen extending therebetween, wherein the distal end comprises at least one aperture; and an inner component comprising a distal end, a proximal end and a lumen extending therebetween, wherein the proximal end comprises at least one opening to receive a finger of a human subject, and further wherein the distal end comprises at least one abrasive head and at least a portion of the abrasive head is configured to protrude past the aperture of the outer shield assembly. In one embodiment, the sample collection device further comprises a locking mechanism to secure the inner component in the outer shield assembly. In one embodiment, the locking mechanism is at or near the proximal end of the outer shield assembly. In one embodiment, the inner component further comprises a tab that is configured to engage the locking mechanism. In another embodiment, the sample collection device further comprises a release mechanism configured to protrude the abrasive head past the aperture of the outer shield assembly. In yet another embodiment, the abrasive head comprises at least one of bristles, foam, abrasive polymer, or adhesive. In still yet another embodiment, the abrasive head is flexible.

In another aspect, the present disclosure provides sample components comprising a flexible member and an abrasive head having a plurality of abrasive surfaces comprising two or more wires wherein the wires comprise abrasive surfaces. In one embodiment, at least one of the wires is a flexible wire. In one embodiment, the abrasive surfaces comprise the sample component comprises at least one of plastic, silicone, multiple bristles, foam, or adhesive. In one embodiment, the abrasive surfaces comprise multiple bristle portions or sections. In one embodiment, at least one or a portion of the flexible wires comprise bristle portions or sections. In another embodiment, at least one of the wires are configured to expand once inserted into a patient cavity.

In another aspect, the present disclosure provides methods of collecting a biological sample using a sample collection device disclosed herein comprising inserting the outer shield assembly and the flexible tip portion into a patient cavity; pushing the inner component through the lumen of the outer shield assembly to protrude the inner component past the distal end of the outer shield assembly thereby opening the flexible tip portion; inserting a sample component through the lumen of the inner component and outer shield assembly until an abrasive head at a distal end of the sample component extends past the distal end of the inner component; and collecting a biological sample from the patient cavity on the sample component.

In another aspect, the present disclosure provides methods of collecting a biological sample comprising inserting a finger of a human subject comprising a sample collection device disclosed herein into a patient cavity; inserting a sample component through the channel on the inner dorsal surface of the device until an abrasive head at a distal end of the sample component extends past the distal end of the device; and collecting a biological sample from the patient cavity on the sample component.

In another aspect, the present disclosure provides methods of collecting a biological sample comprising inserting at least one finger of a human subject comprising a glove-based sample collection device disclosed herein; pushing the inner layer through the outer layer thereby exposing the sample component on the distal tip of the finger portion; and collecting a biological sample from the patient cavity on the sample component.

In another aspect, the present disclosure provides methods of collecting a biological sample comprising inserting at least one finger of a human subject comprising a glove-based sample collection device disclosed herein into a patient cavity; pushing the sample component through the lumen of the channel until an abrasive head at a distal end of the sample component extends past the second channel opening; and collecting a biological sample from the patient cavity on the sample component.

In another aspect, the present disclosure provides methods of collecting a biological sample comprising positioning a sample collection device disclosed herein into or near a patient cavity with the flexible sample component configured away from the patient cavity such that the abrasive head portion is not exposed; pushing the inside surface of the distal end of the flexible sample component into the patient cavity to expose the abrasive head portion; collecting a biological sample from the patient cavity on the abrasive head portion; pulling on the removal component to retract the abrasive head portion so that the abrasive head portion is not exposed; and removing the sample collection device from the patient cavity.

In yet another aspect, the present disclosure provides methods of collecting a biological sample using a sample collection device disclosed herein, comprising inserting the outer shield assembly into a patient cavity; inserting the inner component through the lumen of the outer shield assembly; engaging the inner component with the adapter to guide the inner component; twisting the inner component until an abrasive head at a distal end of the inner component extends past the distal end of the outer shield assembly component; and collecting a biological sample from the patient cavity on the sample component.

In yet another aspect, the present disclosure provides methods of collecting a biological sample using a sample collection device disclosed herein comprising inserting the outer shield assembly into a patient cavity; positioning the aperture of the outer shield assembly on or near a sample for collection; twisting the inner component to expose the at least one abrasive head portion past the aperture of the outer shield assembly; and collecting the biological sample from the patient cavity on the abrasive head portion.

In one embodiment, at least a portion of a sample collection device is pre-lubricated prior to insertion into a patient cavity.

In one embodiment, the methods of collecting a biological sample further comprise imparting a rotational motion on the sample component and/or abrasive head to collect the biological sample from the patient cavity.

### BRIEF DESCRIPTION OF THE DRAWINGS

FIG. 1 is a front isometric view of outer and inner assemblies of a sample device configured in accordance with an embodiment of the present technology.

FIG. 2 is a front isometric view of the sample collection device of FIG. 1 in an assembled configuration with a sample component inserted therethrough in accordance with an embodiment of the present technology.

FIG. 3 is a side view of the assembled sample collection device of FIG. 2 illustrating example dimensions in accordance with an embodiment of the present technology.

FIG. 4A is an isometric view of a finger worn sample collection device configured in accordance with another embodiment of the present technology.

FIG. 4B is an isometric view of a finger worn sample collection device configured in accordance with another embodiment of the present technology.

FIG. 4C is an isometric view of a finger worn sample collection device configured in accordance with another embodiment of the present technology.

FIG. 4D is an end view of a finger worn sample collection device configured in accordance with another embodiment of the present technology.

FIG. 5A is a top view of the finger worn sample collection device of FIG. 4.

FIG. 5B is a bottom view of the finger worn sample collection device of FIG. 4.

FIG. 6A is a bottom view of the finger worn sample collection device of FIG. 4 with a sample component inserted therethrough.

FIG. 6B is a side view of the finger worn sample collection device of FIG. 4 with the sample component and a user finger inserted therethrough.

FIG. 7A is a side view of the finger worn sample collection device of FIG. 4 with a sample component inserted therethrough illustrating example dimensions of the device and component.

FIG. 7B is a side view of the finger worn sample collection device of FIG. 4 with the sample component inserted past a distal end of the device illustrating example dimensions of the device and component.

FIG. 7C is a side view of the finger worn sample collection device of FIG. 7B with a user finger inserted therethrough.

FIG. 8 is a side view of a portion of a sample component configured in accordance with an embodiment of the present technology.

FIG. 9 is a side view of a portion of a sample component configured in accordance with another embodiment of the present technology.

FIG. 10 is a side view of a portion of a sample component configured in accordance with another embodiment of the present technology.

FIG. 11 is a side view of a portion of a sample component configured in accordance with another embodiment of the present technology.

FIG. 12 is a side view of a portion of a sample component configured in accordance with another embodiment of the present technology.

FIG. 13 is a side view of a portion of a sample component configured in accordance with another embodiment of the present technology.

FIG. 14 is a side view of a portion of a sample component configured in accordance with another embodiment of the present technology.

FIG. 15 is a side view of a portion of a sample component configured in accordance with another embodiment of the present technology.

FIG. 16 is a side view of a portion of a sample component configured in accordance with another embodiment of the present technology.

FIG. 17 is a side view of a portion of a sample component configured in accordance with another embodiment of the present technology.

FIG. 18 is a side view of a portion of a sample component configured in accordance with another embodiment of the present technology.

FIG. 19 is a side view of a portion of a sample component configured in accordance with another embodiment of the present technology.

FIG. 20A is a side view of a portion of a sample component configured in accordance with another embodiment of the present technology.

FIG. 20B is a side view of the sample component of FIG. 20A with the multiple abrasive surfaces expanded.

FIG. 20C is a side view of the distal end of the inner component of the sample collection device of FIG. 1 with the sample component of FIG. 20A collapsed within the lumen of the inner component.

FIG. 20D is a side view of the distal end of the inner component of the sample collection device of FIG. 1 with the sample component of FIG. 20A protruding past the distal end of the inner component having bristles of the abrasive head portion of the sample component expanded outwardly.

FIG. 21 is a side view of a portion of a sample component configured in accordance with another embodiment of the present technology.

FIG. 22A is a side view of a glove-based sample collection device configured in accordance with another embodiment of the present technology.

FIG. 22B is a side view of the glove-based sample collection device of FIG. 22A with a sample component exposed.

FIG. 23 is a bottom view of the glove-based sample collection device illustrating certain features of the device configured in accordance with an embodiment of the present technology.

FIG. 24 is a bottom view of the glove-based sample collection device illustrating certain features of the device configured in accordance with an embodiment of the present technology.

FIG. 25 is a side view of a glove-based sample collection device configured in accordance with another embodiment of the present technology.

FIG. 26 is a bottom view of the glove-based sample collection device of FIG. 25 illustrating certain features of the device configured in accordance with an embodiment of the present technology.

FIG. 27A is a side view of a glove-based sample collection device illustrating certain outer features of the device configured in accordance with an embodiment of the present technology.

FIG. 27B is a side view of a glove-based sample collection device illustrating certain features of the outer and inner layers of the device configured in accordance with an embodiment of the present technology.

FIG. 27C is a side view of a glove-based sample collection device illustrating certain features of the inner layer and sample component protruding past the outer layer of the device configured in accordance with an embodiment of the present technology.

FIG. 28A is a side view of a sample component in accordance with an embodiment of the present technology in accordance with an embodiment of the present technology.

FIG. 28B is a side view of an expanded sample component in accordance with an embodiment of the present technology.

FIG. 29A is a side view of the outside of an assembled sample collection device configured in accordance with an embodiment of the present technology.

FIG. 29B is a cut away view of the assembled sample collection device of FIG. 29A in accordance with an embodiment of the present technology.

FIG. 30A is a side view of a sample collection device having a threaded component and push button activator configured in accordance with an embodiment of the present technology.

FIG. 30B is a side view of the assembled sample collection device of FIG. 30A in an activated configuration in accordance with an embodiment of the present technology.

FIG. 31A is a side view of a sample collection device having a threaded component and push button activator configured in accordance with an embodiment of the present technology.

FIG. 31B is a cut away view of the assembled sample collection device of FIG. 31A in accordance with an embodiment of the present technology.

FIG. 32A is a side view of a sample collection device having a threaded component and push button activator configured in accordance with an embodiment of the present technology.

FIG. 32B is a cut away view of the assembled sample collection device of FIG. 32A in accordance with an embodiment of the present technology.

FIG. 33A is a side view of a flexible finger worn sample collective device in accordance with an embodiment of the present technology.

FIG. 33B is a side view of the flexible finger worn sample collection device of FIG. 33A configured with an exposed abrasive head in accordance with an embodiment of the present technology.

FIG. 34A is a rear isometric view of outer shield assembly and inner component of a sample collection device configured in accordance with an embodiment of the present technology.

FIG. 34B is a rear isometric view of the finger worn sample collection device of FIG. 34A in an assembled configuration with the inner component inserted therethrough in accordance with an embodiment of the present technology.

FIG. 35A is a side view of a push-and-twist finger worn sample collection device in accordance with an embodiment of the present technology.

FIG. 35B is a side view of a push-to-expand finger worn sample collection device in accordance with an embodiment of the present technology.

### DETAILED DESCRIPTION

The present technology describes various embodiments of sampling collection devices and associated systems and methods for collecting samples (e.g., tissue samples) of mammals or other animals in the diagnostic or other contexts. For example, various sampling (e.g., cytology) collection devices, features, and methods for their use are described herein. The various devices and methods generally employ reduced or minimally invasive technologies in order to collect cytology samples from, for example, the gastrointestinal body of a patient. In several embodiments, for example, a sampling collection device can be inserted into a cavity, e.g., through an anal canal and into a rectum, and introduced to a nearby site (e.g., the cervix, colon, etc. of a patient) that may be investigated for cellular inconsistencies. The present technology also describes systems and methods for collecting samples using rapid collection procedures.

Many diagnostic procedures have been developed over the years which have diminished the need for more severe procedures. Particularly, the Pap Smear screening has paved the way for cervical cancer prevention. Studies have shown that the risk of developing invasive cervical cancer is three to ten times greater in women who have not been screened, demonstrating the importance of these tests and accurate sample collection.

Similarly, for colon cancer screening, a colonoscopy is the state of the art procedure. However, the technique is costly, requires sedation and is lengthy which increases the cost and burden on the healthcare system. Novel procedures such as virtual colonoscopy done via a CT or MRI scan has gained traction but is usually not as widely available or covered by healthcare providers (e.g., health insurance). Additionally, if a virtual procedure detects any abnormalities, generally a conventional colonoscopy must be performed.

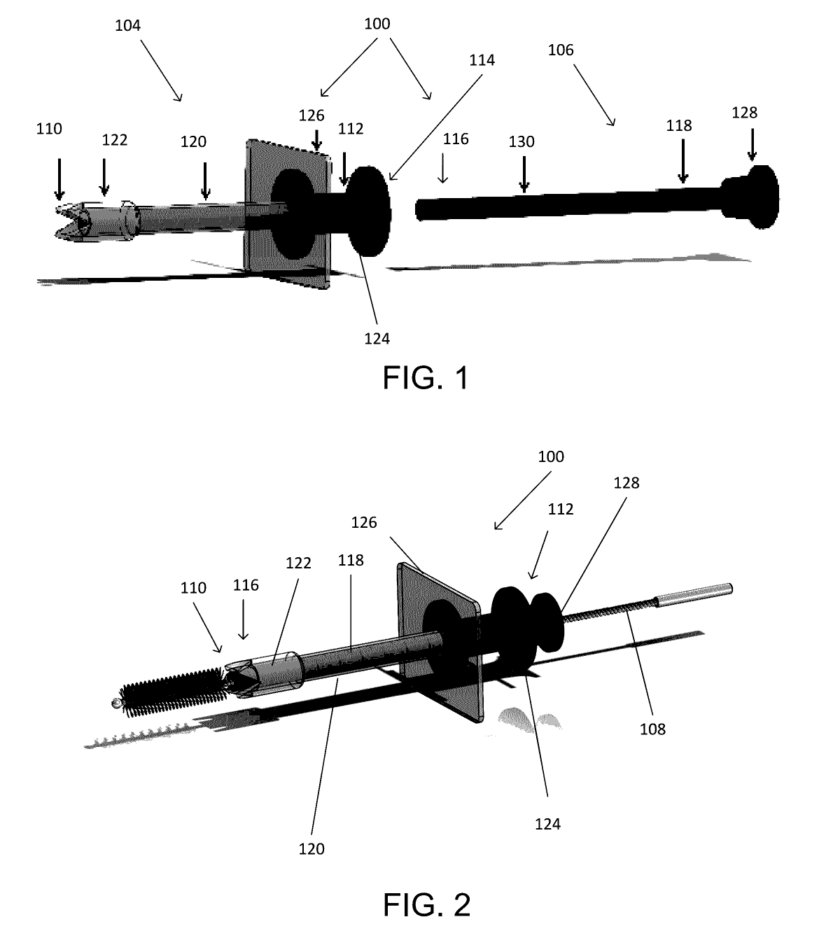

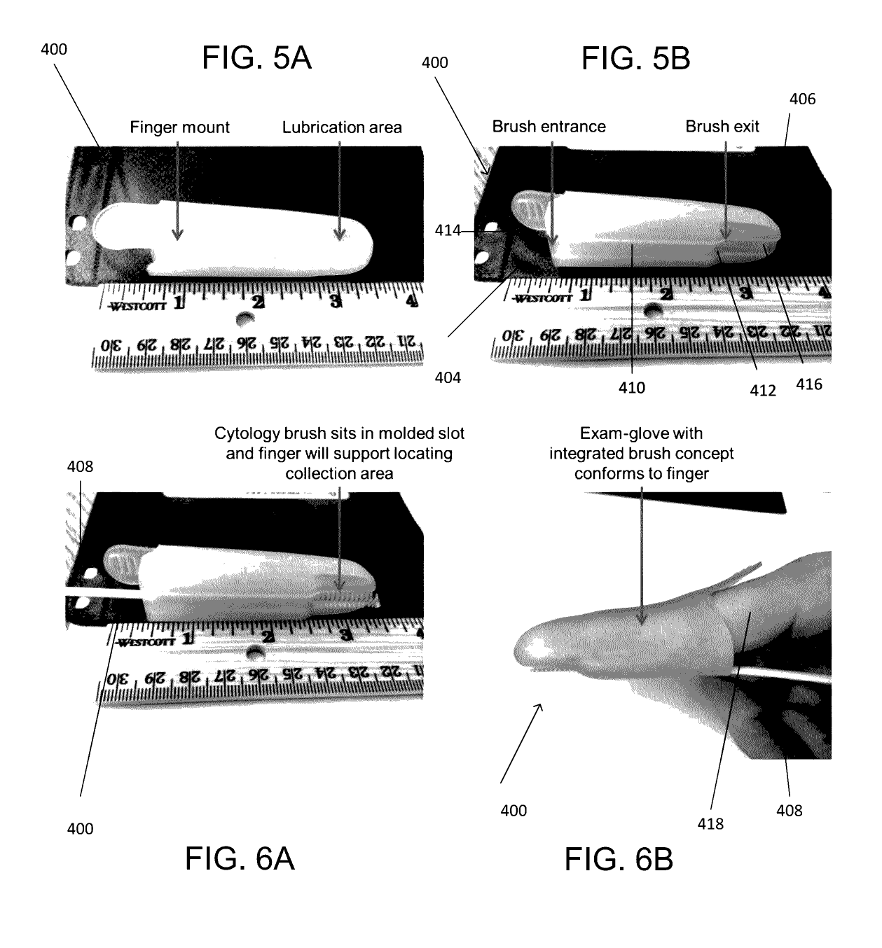

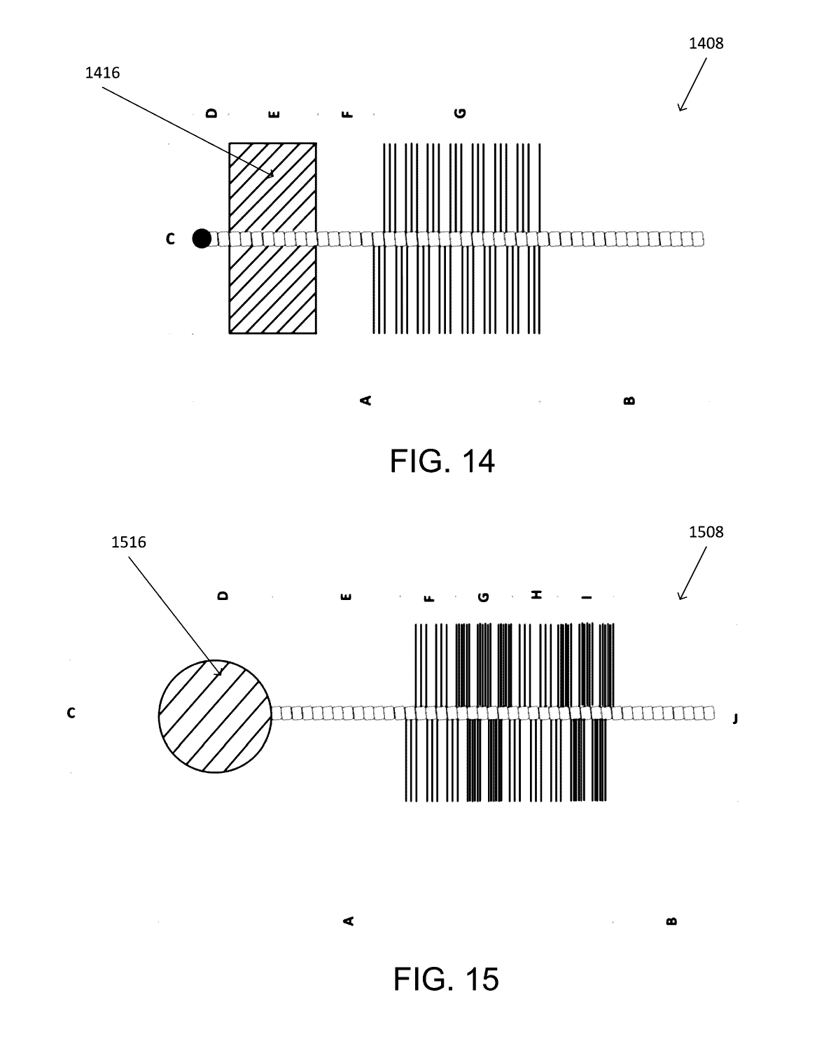

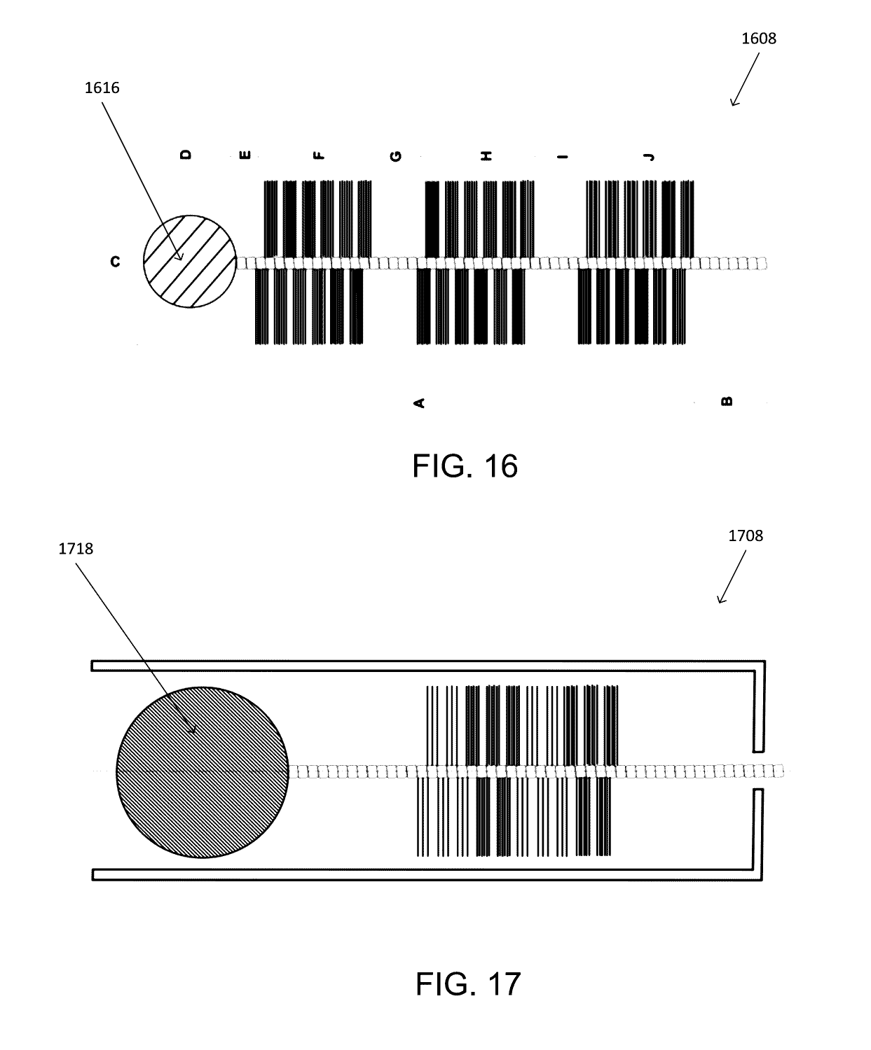

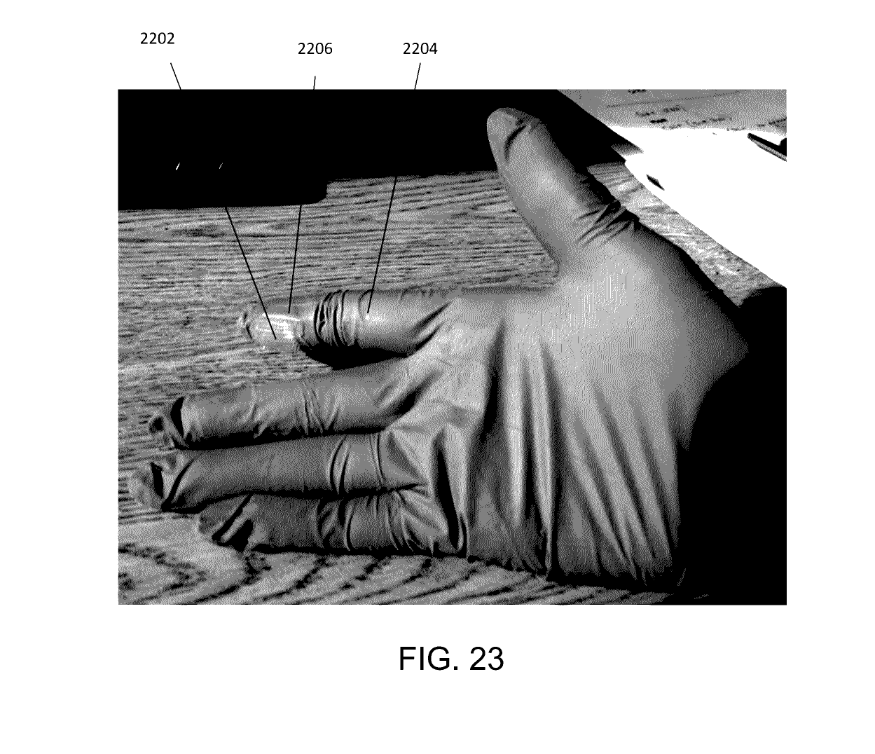

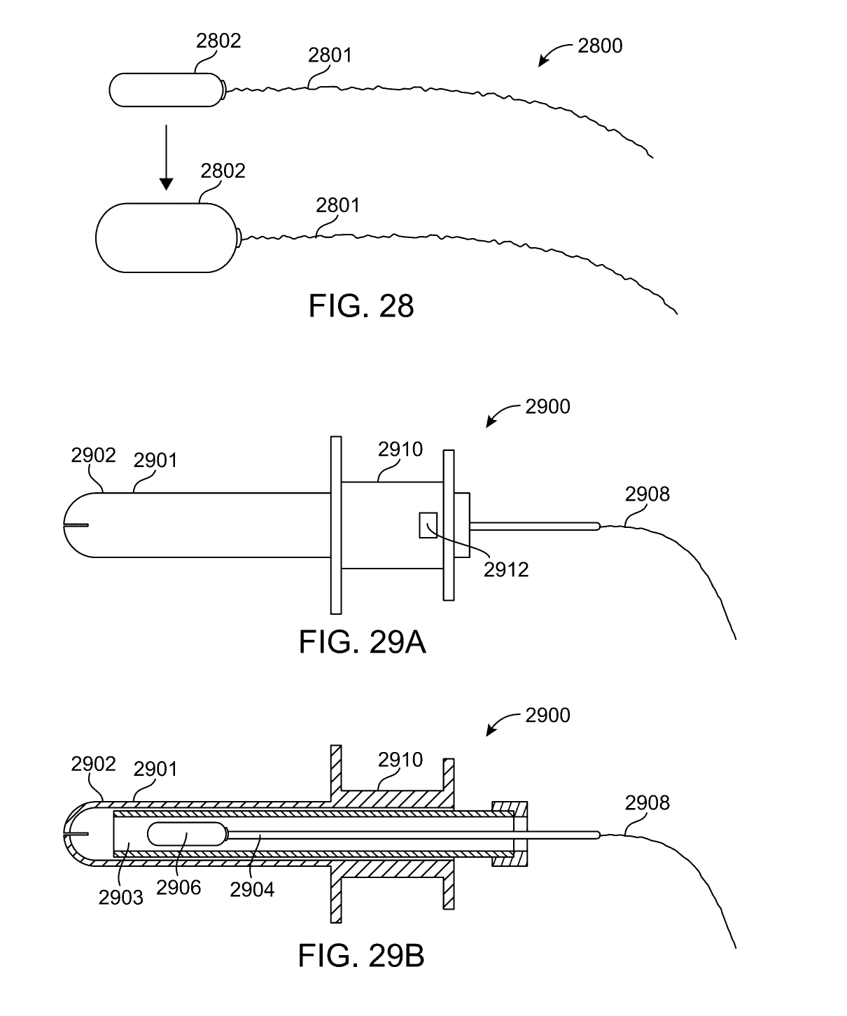

Certain details are set forth in the following description and in FIGS. 1-35 to provide a thorough understanding of various embodiments of the present technology. Other details describing well-known structures and systems often associated with sample collection devices however, are not set forth below to avoid unnecessarily obscuring the description of the various embodiments of the present technology.

Many of the details, dimensions, angles and other features shown in FIGS. 1-35 are merely illustrative of particular embodiments of the present technology. Accordingly, other embodiments can include other details, dimensions, angles and features without departing from the spirit or scope of the present invention. In addition, those of ordinary skill in the art will appreciate that further embodiments of collection devices described herein can be practiced without several of the details described below. Various embodiments of the present technology can also include structures other than those illustrated in the Figures and are expressly not limited to the structures shown in the Figures. Moreover, the various elements and features illustrated in the Figures may not be drawn to scale.

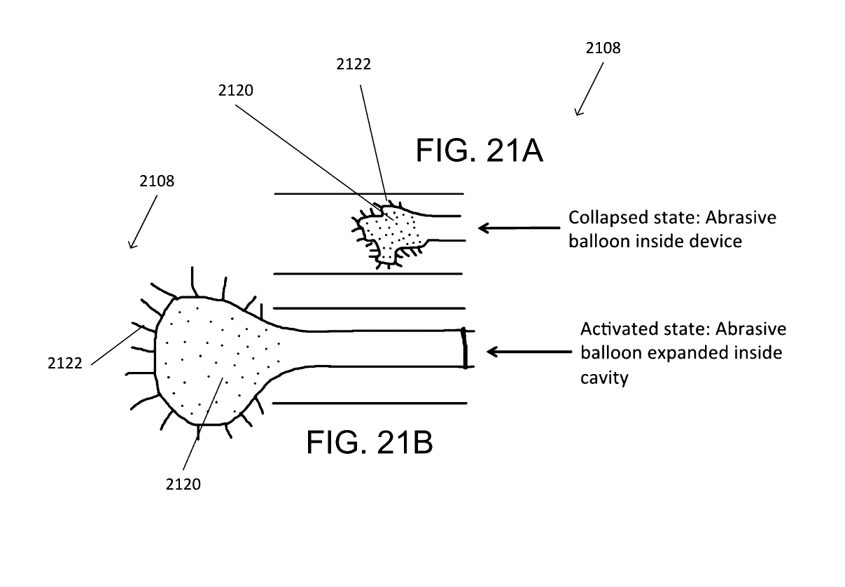

In the Figures, identical reference numbers identify identical or at least generally similar elements. To facilitate the discussion of any particular element, the most significant digit or digits of any reference number refers to the Figure in which that element is first introduced. For example, element 110 is first introduced and discussed with reference to FIG. 1 and element 2110 is first introduced and discussed with reference to FIG. 21.

### Sample Collection Device

FIG. 1 is a front isometric view of a sample collection device 100 configured in accordance with an embodiment of the present technology. The sample collection device 100 (e.g., an insertion device) is configured to protect or shield a sample component 108 (e.g., an abrasive component) and can include an outer component or assembly (also referred to as an outer shield assembly) 104 and an inner component or assembly 106. The outer and inner assemblies 104,106 can be separate components configured to be removably secured or attached together as described in more detail below. In other embodiments, various components of the sample collection device 100 can be monolithically or integrally formed together as a single component. Both the outer and inner assemblies 104,106 can be formed from suitable flexible or rigid materials.

The sample collection device 100 includes a proximal end and a distal end, for guiding the sample component 108 into and through a cavity (e.g., an orifice or lumen) of a patient. The outer shield assembly 104 includes a distal end 110 opposite a proximal end 112. In certain embodiments, the sample collection device 100 can be configured to be small enough to held with one hand of a user (e.g., by a doctor, physician, surgeon, or other medical or clinical practitioner). The proximal end 112 of the outer shield assembly 104 includes an opening 114 for receiving a distal end 116 of the inner component 106 to be inserted therethrough. A first body portion (i.e., lumen) 118 of the inner component 106 is configured to travel or extend through a second body portion (i.e., lumen) 120 of the outer shield assembly 104. For example, the second body portion 120 can be an outer tube (e.g., conduit, sheath, etc.) and the first body portion 118 can be an inner tube (e.g., conduit, sheath, etc.) configured to be inserted into and extend at least partially through the outer tube as illustrated and described in more detail with respect to FIG. 2.

The distal end 110 of the outer shield assembly 104 includes a movable (e.g., deformable or resilient) tip portion 122. For example, the tip portion 122 can be made from a flexible or other suitable material which can aid in inserting or maneuvering the sample collection device 100 into or through variously shaped anatomical cavities, openings, and conduits. The tip portion 122 can open (e.g., by deforming) when the inner component 106 is inserted though the outer shield assembly 104 until the distal end 116 of the inner component 106 protrudes from the distal end 110 of the outer shield assembly. By opening the tip portion 122, another component (e.g., the sample component 108) may be inserted through the sample collection device 100. The tip portion 122 may be unitarily or monolithically formed with the rest of the outer shield assembly 104 as a single component. In other embodiments, the tip portion 122 and the outer shield assembly 104 can be two separate components.

The second body portion 120 of the outer shield assembly 104 can be connected or coupled to a handle 124. The handle 124 can have a spool-shaped body or other suitable configuration with the opening 114 or channel extending therethrough between first and second ends of the handle 124. The handle 124 may be formed from a flexible or rigid material. The handle 124 may be a single, unitary (e.g., monolithically formed) piece or composed of several pieces assembled together.

A protective barrier 126 can be secured or coupled to a distal end of the handle 124 between the distal and proximal ends 110,112 of the outer shield assembly 104. The protective barrier 126 can prevent the sample collection device 100 from being inserted too deep into a cavity or orifice (e.g., the protective barrier 126 can be greater in diameter than the cavity or orifice to prevent further insertion of the outer shield assembly 104). The protective barrier 126 can also provide protection (e.g., contamination protection) against fluids that may escape or leak out of the cavity or orifice and flow around the circumference of second body portion 120. As illustrated, in some embodiments, the protective barrier 126 can have a rectangular cross-sectional shape. In other embodiments, the protective barrier 126 can have a circular or other suitable cross-sectional shape. The protective barrier 126 can be formed from a flexible (e.g., silicone) or rigid material.

As described above, the first body portion 118 of the inner assembly 106 can be an inner tube (e.g., conduit, sheath, etc.) having proximal and distal ends. The first body portion 118 can be made from a flexible or rigid material. In some embodiments, the first body portion 118 can include one or more slots or grooves 130 to increase flexibility of the first body portion 118. The first body portion 118 can also include a stopper component 128 configured to be secured or attached to a proximal end of the first body portion 118. The stopper component 128 can be configured such that it cannot be inserted into the opening 114. For example, the stopper component 128 can have a diameter greater than the diameter of the opening 114. This can prevent the inner component 106 from being inserted further into the outer shield assembly 104 when the stopper component 128 contacts a proximal end of the handle 124.

FIG. 2 illustrates an assembled configuration of the sample collection device 100. During assembly, the inner component 106 is inserted into the opening 114 at the proximal end 112 of the outer shield assembly 104. The stopper component 128 can prevent the first body portion 118 of the inner component 106 from being inserted too deep or far into the cavity of the patient. For example, the stopper component 128 provides a physical and visual signal to a user that the inner component 106 is fully inserted into the outer shield assembly 104 when a distal end of the stopper component 128 reaches and contacts the proximal end 112 of the outer shield assembly 104 (e.g., the handle 124) and cannot be moved farther toward the distal end 110 of the outer shield assembly 104. The stopper component 128 can also serve to signal to the user when the sample component 108 can be inserted through the inner component 106 (e.g., when the inner component 106 has been fully inserted into the outer shield assembly 104).

During assembly, as the inner component 106 is inserted through the handle 124 and toward the distal end 110 of the outer shield assembly 104, the tip portion 122 of the outer shield assembly 104 will begin to open as the distal end 116 of the inner component 106 is pushed through the tip portion 122. Once the inner component is fully inserted, whereby the stopper component 128 contacts the handle 124, the tip portion 122 is fully opened (e.g., spread or deformed outward). Such features prevent the sample component 108 from directly touching or contacting the tip portion 122 when inserted through the inner and outer assemblies 106,104. The tip portion 122 may be contaminated with fluids or other tissue when the outer shield assembly 104 was first inserted into the cavity or other orifice of the patient. Further, the tip portion 122 may be covered with a lubricant to aid inserting the outer shield assembly 104 that may potentially contaminate any potential sample collected with the sample component 108. The shape or contour of the distal end 116 of the inner component 106 can be rounded, smooth, and/or curved to minimize any sharp edges to facilitate insertion of the inner component 106 through the outer shield assembly 104.

The outer shield assembly 104 (e.g., the tip portion 122) aids in moving tissue away from a potential sample site prior to inserting the inner component 106. Once the inner component 106 is fully inserted into the outer shield assembly 104 (e.g., the stopper component 128 makes contact with the handle 124), the sample component 108 may be inserted into and through the inner component 106. The sample component 108 is inserted until it exits the distal end 110 of the outer shield assembly 104 near the tip portion 122 and into the patient sample site.

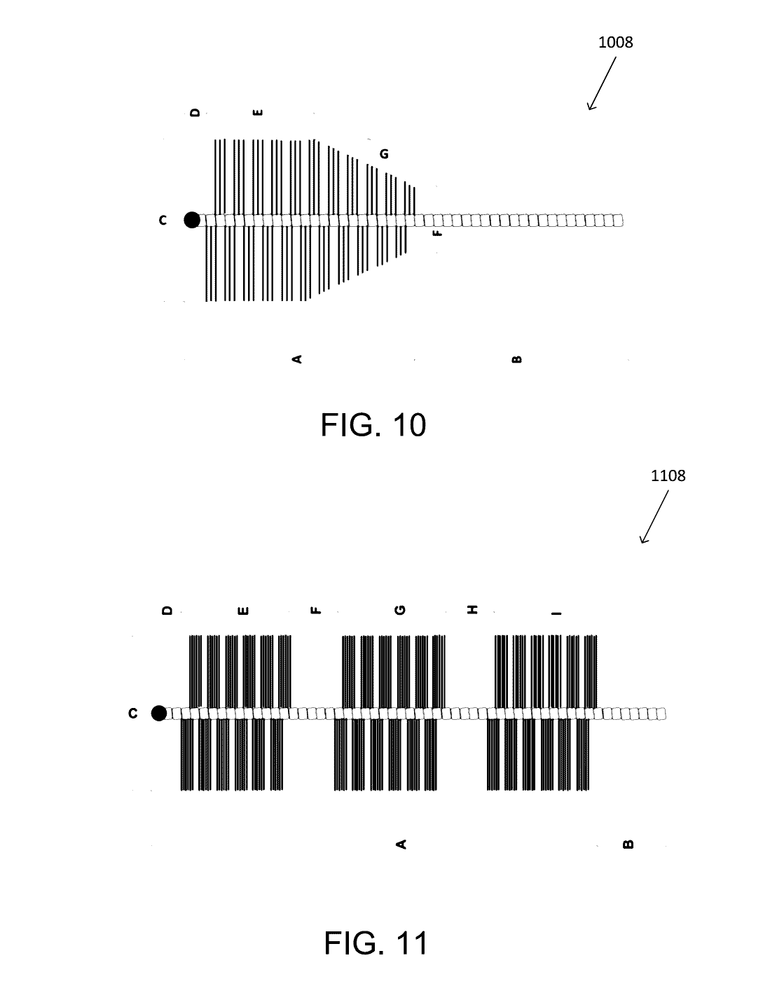

In some embodiments, the sample component 108 can be an abrasive sample component configured to be inserted into a cavity of a patient to a desired sample collection site in the patient through the inner and outer assemblies 106,104 to collect a tissue sample. Example sample components or abrasive head portions that can be used with the sample collection devices described herein are described in more detail below with respect to FIGS. 8-21B and FIG. 28. However, the sample collection devices are not limited to using such sample components. The sample component 108, as shown in FIG. 2, is inserted into the sample collection device 100 through the inner and outer assemblies 106,104. The conduits, tubes, or sheaths of the inner and outer assemblies 106,104 are sized such that the sample component and/or abrasive head portion can move or rotate in a circular or radial direction (e.g., to collect or swab a tissue sample). The sample component 108 can include an abrasive head portion (e.g., a brush or bristles) for collecting a sample.

The interior spaces (e.g., body portions, conduits, sheaths, tubes, the handle 124, the stopper component 128, the protective barrier 126, the tip portion 122, etc.) of the sample collection device 100 can sized to permit passage of other structures and components to be inserted through or along the sample collection device 100. For example, a vacuum tube may be inserted through the sample collection device 100. The vacuum tube may be used to remove liquid, fluid, or other debris (e.g., from the collection site). An irrigation conduit (e.g., for providing liquid, fluid, etc.) to the sample collection site can also be inserted through or along the sample collection device 100 in certain embodiments. In some embodiments, an optical sensor can be used with the sampling device 100 (e.g., with the tip portion 122 or sample component 108) to signal to a user a location of the sample collection device 100 within the patient. In other embodiments, a camera can be used, for example, to provide video of the insertion site. All materials forming the sample collection device 100, sample component 108, and/or other components used with the sample collection device 100 can be biocompatible and approved for use in humans.

Further in some embodiments, the tip portion 122, second body portion 120, protective barrier 126, and/or handle 124 can form a first distinct assembly (e.g., the outer shield assembly 104) and the first body portion 118 and the stopper component 128 can form a second distinct assembly (e.g., the inner component 106). The first and second distinct assemblies can function together and be assembled to form the sample collection device 100 for receiving the sample component 108 or other components.

In other embodiments, the sample collection device 100 includes an integrated hand piece whereby the first body portion 118, the stopper component 128 of the inner component 106 and the sample component 108 (e.g., an abrasive component) are integrated with the tip portion 122, second body portion 120, the protective barrier 126 and the handle 124. This embodiment can control how far the first body portion 118 and/or the integrated sample component 108 are inserted or retracted relative to the outer shield assembly or cavity (e.g., the distance of insertion and retraction). The distance can be controlled by, for example, a slide feature whereby a user can push the slide feature forward to enable insertion and pulls the feature back to enable retraction. The feature can enable the position of the first body portion 118 and/or the integrated sample component 108 to be locked before and/or during sample collection. The sliding feature may reach a point along the handle 124 that restricts the sliding features motion and prevents its return without additional force. For example, it can include an extruded feature that can interfere with or lock the free motion of the sliding feature. In an alternative embodiment, the device can include a mechanism (e.g., a knob that is twisted or turned) which will move the first body portion 118 and/or the integrated sample component 108 forward or backward. The stopper component 128 can have a threaded component which is inserted into the handle 124. By twisting, the threaded component, the stopper component 128 can be moved forward or backward. Other suitable twisting mechanisms can also be used. Another embodiment, includes integrating the sample component 108 with the device 100 such that it may be push fit into the stopper component 128 to temporarily secure the two structures together. However, the sample component 108 can be removed by pulling it away from the stopper component 128. In certain embodiments, the first body portion 118 can be locked in position after the first body portion 118 is inserted to a proper or desired location. Then after the first body portion 118 is secured or locked in position, the sample component 108 can be inserted through the opening 114 and extend through and exit the distal end 110 of the device 100.

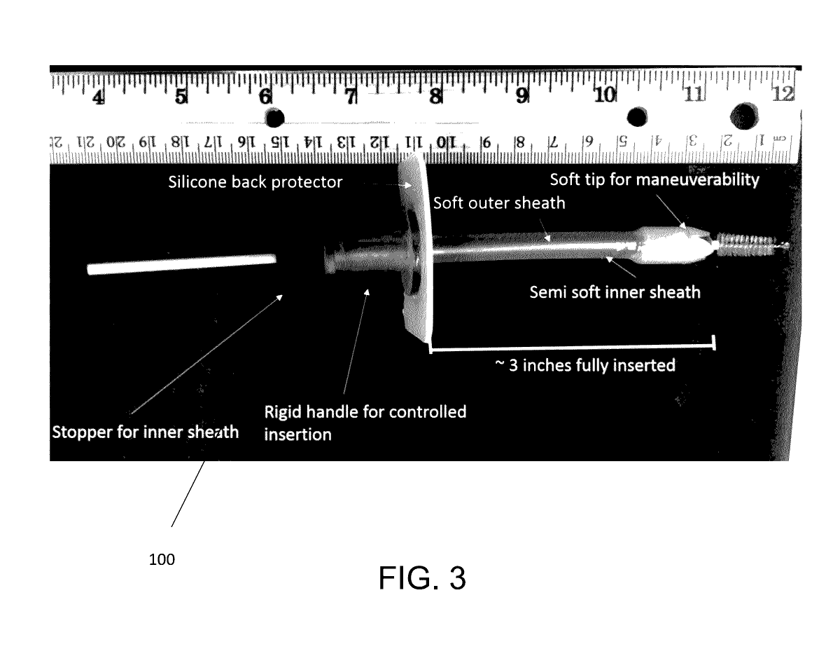

FIG. 3 illustrates example dimensions of various components of the sample collection device 100. In some embodiments, a length of the sample device 100 can be defined as the distance between the proximal end and the distal end (e.g., between the proximal end of the stopper component 128 and the distal end of the tip portion 122 or between the proximal and distal ends of the sample component 108). For example, in some embodiments, the length can be in the range of about 10 cm to about 16 cm and/or any value therebetween. In other embodiments, the length can be in the range of about 6 cm to about 18 cm and/or any value therebetween. In further embodiments, the length can be in the range of about 4 cm to about 20 cm and/or any value therebetween. The length of the sample collection device 100 may vary depending on the required or desired area of sample (e.g., tissue) collection and/or the distance a desired sample collection area is away from a cavity, opening, or orifice the sample collection device 100 can enter through to access the sample collection area. In some embodiments, a length of the outer shield assembly 104 (e.g., between the protective barrier and distal end of the tip portion 122) that is actually inserted into the patient can be about 2 inches, 3 inches, 4 inches, 5 inches, and/or any value therebetween.

FIG. 29 illustrates one embodiment of a sample collection device 2900 configured to have an activator 2912 and threaded fitting 2910 that function to impart rotational motion on the sample component 2906. The sample collection device can comprise an outer shield assembly 2901 with a flexible tip portion 2902, an inner component 2903, a threaded fitting 2910, and an activator 2912. In some embodiments, the activator can be a push button, a switch, or the like. In some embodiments, the sample component 2904 comprises an abrasive head portion 2906, and optionally a removal component 2908. The sample component 2904 can further comprise a threaded portion at a proximal end wherein the threaded portion of the sample component is configured to securably attach to a threaded fitting 2910. Example sample components or abrasive head portions that can be used with the sample collection devices described herein are described in more detail below with respect to FIGS. 8-21B and FIG. 28. However, the sample collection devices are not limited to using such sample components.

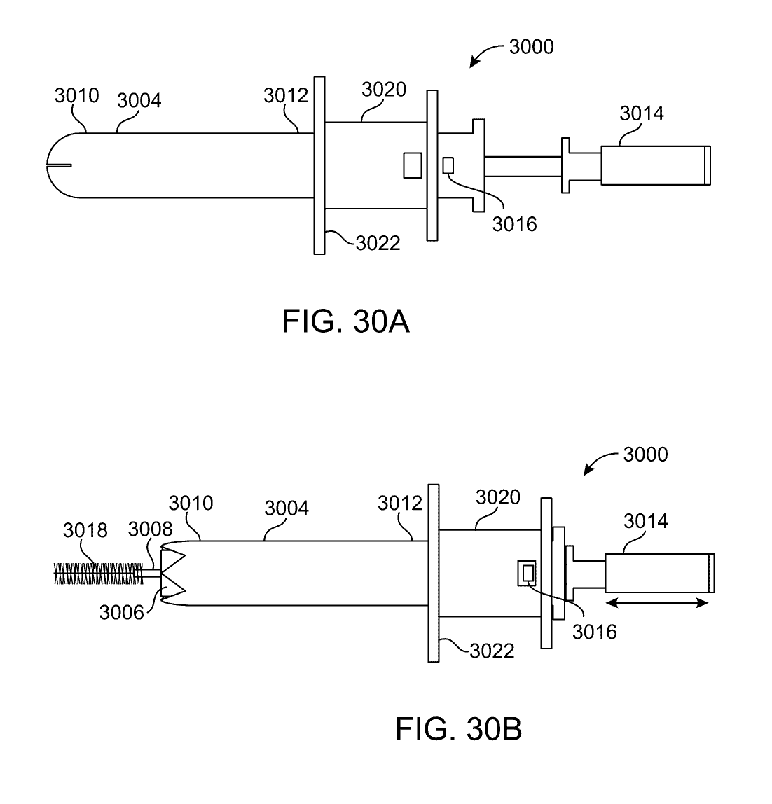

FIG. 30 illustrates another embodiment of a sample collection device 3000 configured to have an activator 3016 and threaded fitting 3014 that function to impart rotational motion on the sample component 3008 and the abrasive head portion 3018. The sample component 3008 can comprises a threaded portion at a proximal end of the sample collection device 3000 which is configured to securably attach to a threaded fitting 3014. In one embodiment, the inner component 3006 securably attaches to the outer shield assembly 3004. In some embodiments, the proximal end 3012 of the outer shield assembly 3004 comprises a handle 3020 and/or a protective barrier 3022. The protective barrier 3022 can be secured or coupled to a distal end of the handle 3020, for example, near the proximal end 3012 of the outer shield assembly 3004. The protective barrier 3022 can prevent the sample collection device 3000 from being inserted too deep into a cavity or orifice (e.g., the protective barrier 3022 can be greater in diameter than the cavity or orifice to prevent further insertion of the outer shield assembly 3004). The sample collection device 3000 can also have an activator 3016 which engages with the handle 3020.

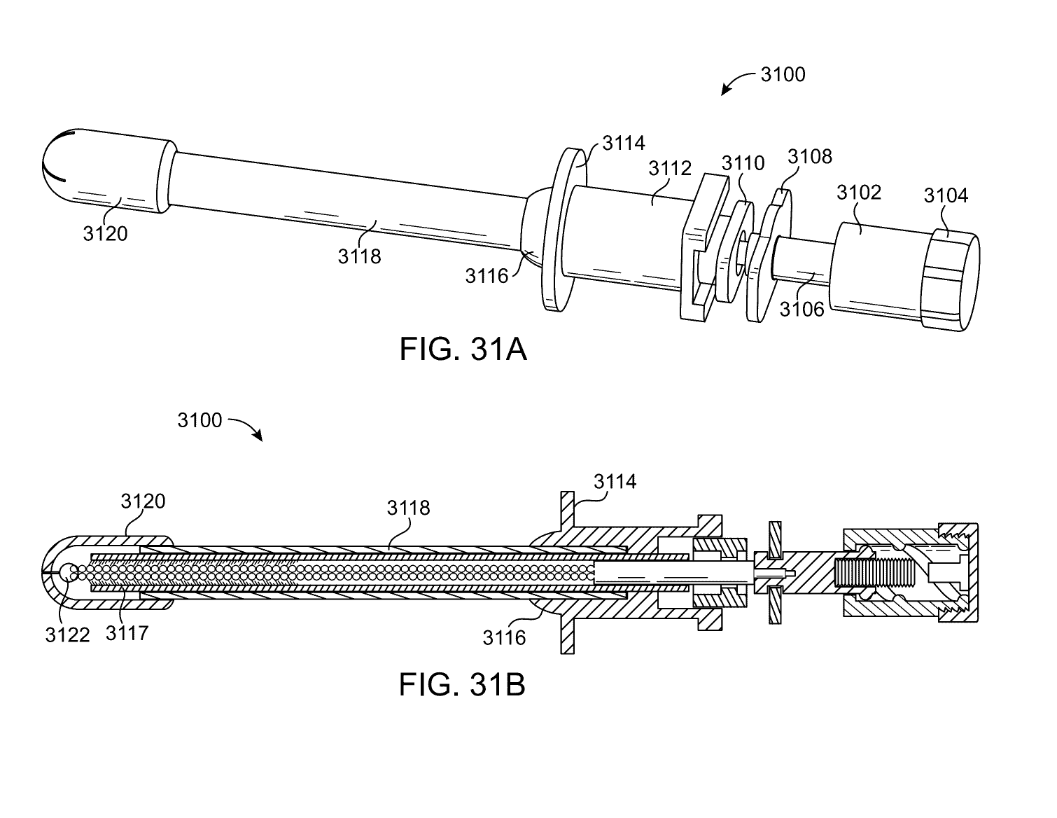

FIG. 31 illustrates yet another embodiment of a sample collection device 3100 configured to have a threaded fitting 3102 and a cap 3104 that function to impart rotational motion on the abrasive head portion 3122 of the sample component. In some embodiments, the cap 3104 is threaded or snapped onto the threaded fitting 3102. In some embodiments, the threaded fitting 3102 is securably attached to the sample collection device 3100 by a housing component 3106. In some embodiments, the inner component 3117 and/or sample component can be releasably secured to the housing component 3106 by a lever 3108. In some embodiments, the proximal end of the sample component or the inner component 3117 comprises a threaded portion that is securably attached to the threaded fitting 3102. The sample collection device 3100 can have a handle 3112 and a protective barrier 3114. In some embodiments, the sample collection device 3100 can have a flexible adaptor 3116 which can conform to the anatomy of a patient. The flexible adaptor 3116 can be attached to the handle 3112 and/or outer shield assembly 3118. The outer shield assembly 3118 can have a flexible tip portion 3120 on the distal end. The flexible tip portion 3120 can be greater in diameter than the outer shield assembly 3118. The flexible tip portion 3120 can be unitarily or monolithically formed with the outer shield assembly 3118. The abrasive head portion 3122 of the sample collection device 3100 can rest inside the flexible tube portion 3122 prior to use such that the abrasive head portion 3122 does not extend out of the sample collection device 3100.

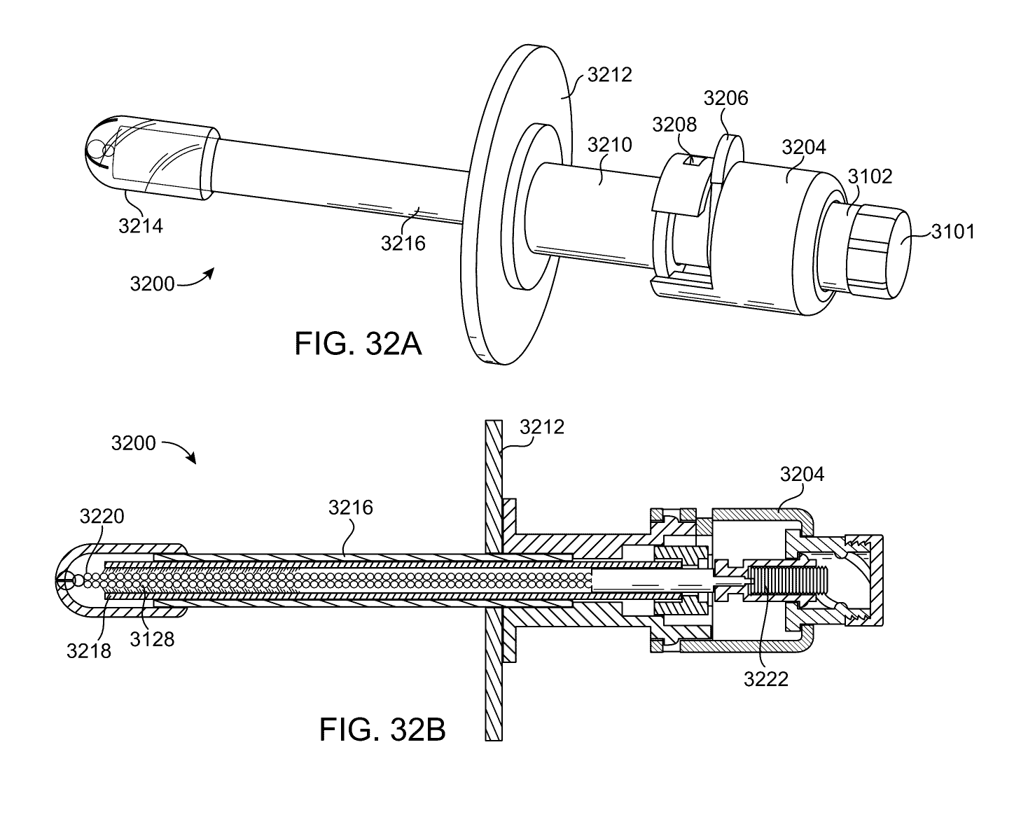

FIG. 32 illustrates yet another embodiment of a sample collection device 3200 configured to have a threaded fitting 3204 and a cap 3201 that function to impart rotational motion on the abrasive head portion 3220 of the sample component 3218. In some embodiments, the cap 3201 is threaded or snapped onto the threaded fitting 3204. The sample collection device 3200 can further comprise an activator 3202, which can be a push button, a switch, or the like. In some embodiments the sample collection device 3200 comprises a lever 3206 that functions to securably attach or release the attachment component 3208 which attaches the threaded fitting 3204 to the outer shield assembly 3218. The sample collection device 3200 can have a handle 3210 and a protective barrier 3212. The outer shield assembly 3216 can have a flexible tip portion 3214 on the distal end. The flexible tip portion 3214 can be unitarily or monolithically formed with the outer shield assembly 3218. The abrasive head portion 3220 and the inner component 3218 of the sample collection device 3200 can rest inside the flexible tube portion 3214 prior to use such that the abrasive head portion 3220 and the inner component 3218 do not extend out of the sample collection device 3200 until the sample collection device 3200 is positioned appropriately for collecting a sample.

Method of Collection with Sample Collection Device

An example method of collecting a sample (e.g., a tissue sample) with the sample collection device 100 can include one or more of the following steps in accordance with an embodiment of the present technology. The sample collection device 100 can be removed from a packaging or other wrapping that it is stored in. The sample collection device 100 can be pre-lubricated or a physician or other user can lubricate the outer shield assembly 104 (e.g., the second body portion 120 and/or the tip portion 122) before inserting the outer shield assembly 104 into the patient.

The sample collection device 100 (e.g., the outer assembly 104) can be inserted into the patient cavity or orifice until the protective barrier 126 is touching or contacting the patient's body. Next, the inner component 106 is inserted into the outer assembly 104 through the opening 114 until the stopper component 128 contacts the handle 124 and pushes the tip portion 122 (e.g., end flaps) outward. The first body portion 118 of the inner component 106 is sized such that when the stopper component 128 contacts the handle 124, the distal end of the first body portion 118 pushes open or otherwise spreads outwardly the tip portion 122.

The sample component 108 (e.g., brush or other abrasive head portion) is then inserted into a proximal opening of the stopper component 128 and inserted through the inner and outer assemblies 106,104 until a head portion (e.g., abrasive part) of the sample component 108 extends past a distal end of the tip portion 122. The sample component 108 can be pre-marked with dashes or other marks along its body or shaft portion. The marks can indicate the location or distance of the sample component 108 (e.g., how far it extends into the patient or past the distal end of the tip portion 122). For example, a user can insert the sample component 108 a desired length into the patient and/or past the distal end of the tip portion 122 based on the location of the marks and/or which marks are still visible.

If any resistance is met while inserting the assemblies or components, the sample collection device 100 (the outer shield assembly 104, inner component 106, and/or sample component 108) may be repositioned (e.g., removed, re-inserted, moved, etc.) accordingly as human and other animal anatomy may vary (e.g., cavity shape may change). The sample component 108 can then be rotated to collect the sample from the desired sample site within the patient. Once the sample is collected, the sample component 108 can be removed from device 100 (e.g., pulled out). After the sample component 108 is removed, the sample collection device 100 (e.g., the inner and outer assemblies 106,104) may be removed and discarded. In some embodiments, the sample collection device 100 (e.g., the inner and outer assemblies 106,104) can be washed or decontaminated for reuse.

### Finger Worn Sample Collection Device

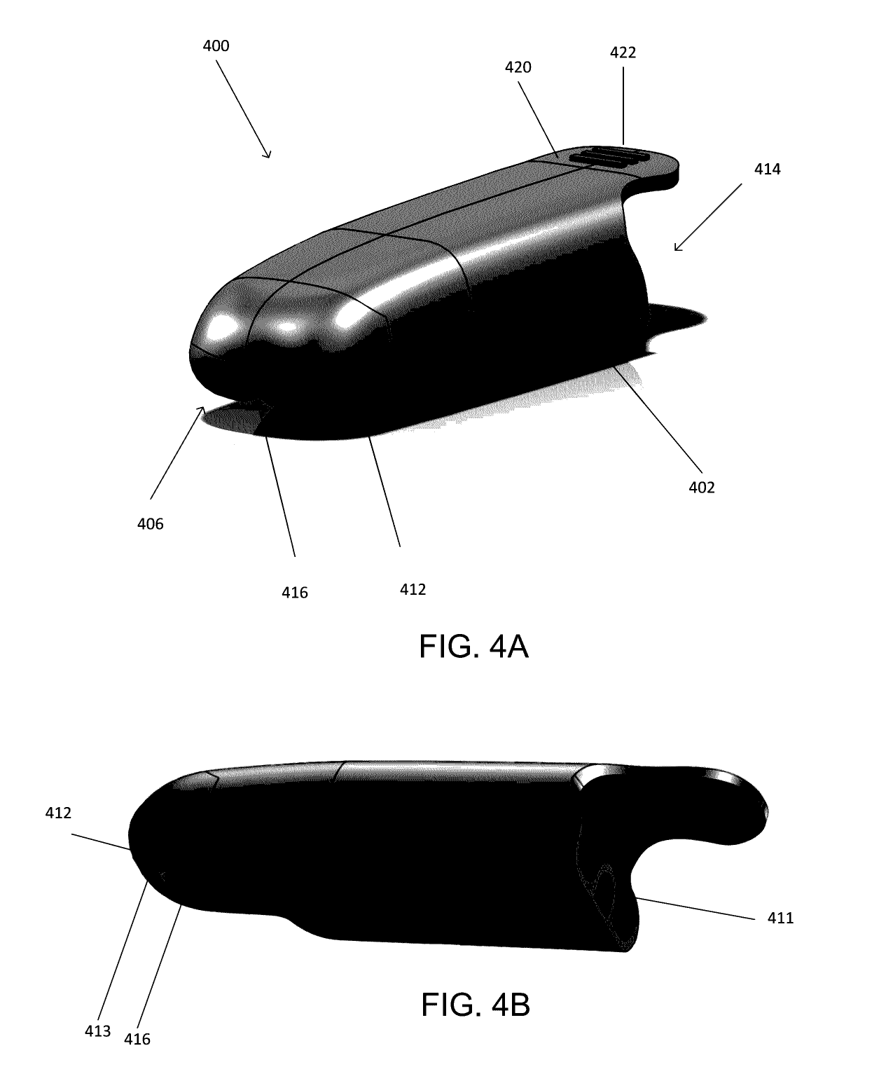

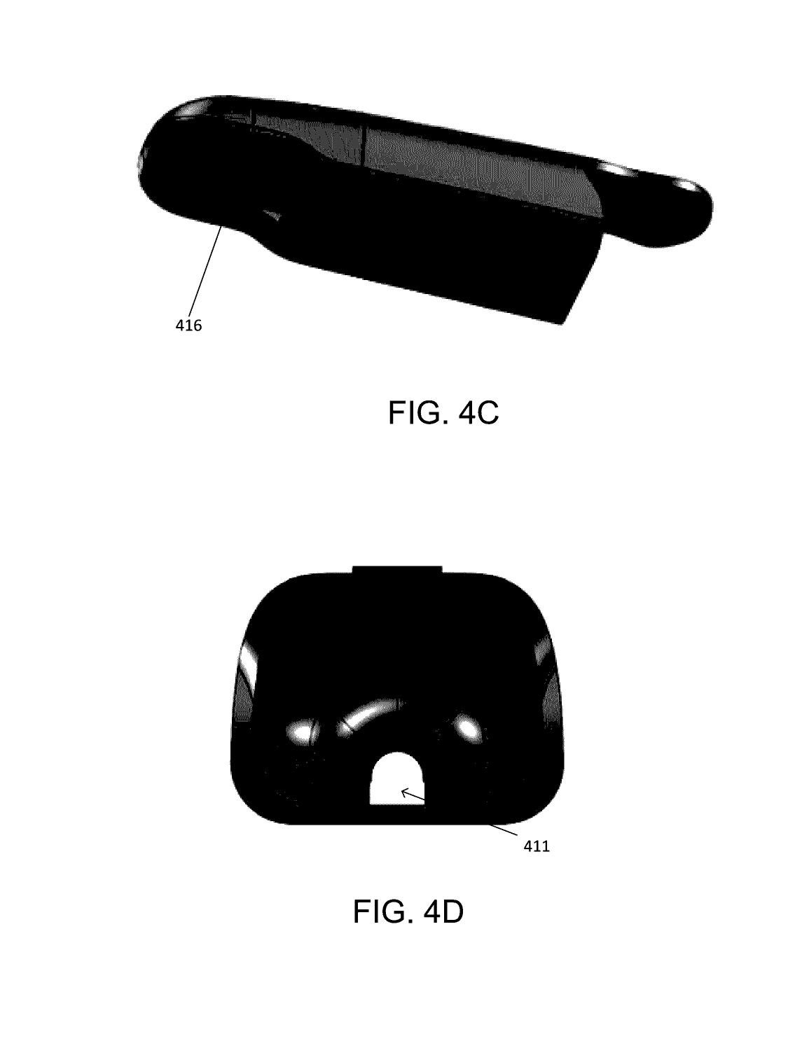

FIGS. 4A-4D illustrate various isometric views and an end view, respectively, of a sample collection device 400 configured in accordance with another embodiment of the present technology. The sample collection device 400 is configured to be worn or placed onto a finger (e.g., the index finger) of a user. Referring to FIGS. 4A-4D together, the sample collection device 400 includes an outer shell 402 (e.g., body, case, mount, etc.) shaped and sized to conform to a user's finger. The shell 402 includes a proximal end 404 and a distal end 406. The proximal end 404 has an opening 414 configured to receive the user's finger 418 (FIGS. 6A and 6B). The device 400 has a channel 410 (e.g., a hollow channel, conduit, or tube) that extends from the proximal end 404 to the distal end 406 along a lower or bottom portion of the shell 402 within an interior or cavity of the shell 402. In some embodiments, the channel 410 can share a wall or surface with an interior portion of the shell 402 (e.g., a bottom or lower surface). The channel 410 can be completely enclosed within the interior of the shell 402. In other embodiments, the channel 410 can be open to the interior (e.g., cavity) of the shell 402.

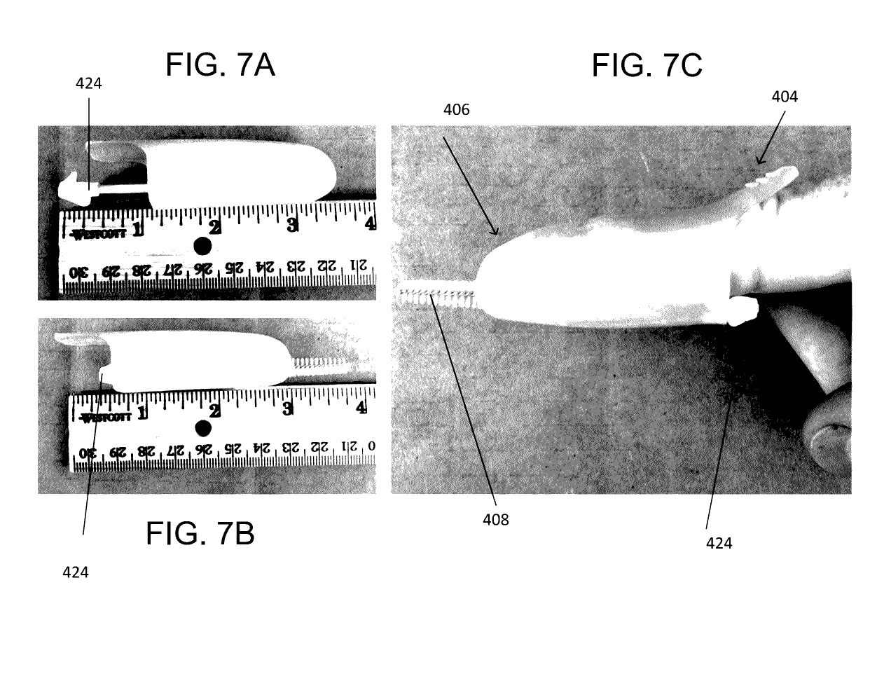

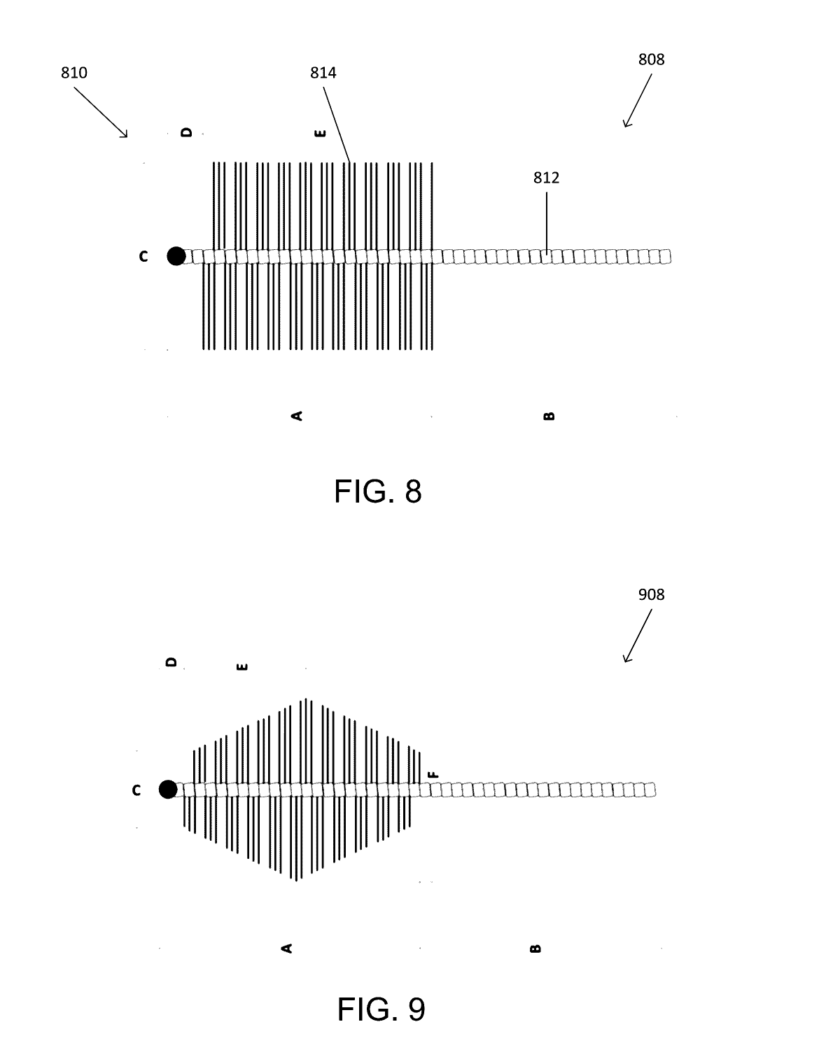

The channel 410 may house and receive a sample component 408 (e.g., an abrasive head portion) configured to extend therethrough and be inserted through a proximal opening (e.g., the opening 414 and/or a proximal opening of the channel 410) and past (e.g., exit) a distal opening 412 of the shell 402. In some embodiments, the sample component 408 can also extend past the distal end 406 of the shell 402 to reach deeper or farther into the cavity of a mammal or other animal (FIGS. 7A-7C). The channel 410 can have a cylindrical, cuboid, prismatic, or other suitable cross-sectional shape.

A bottom or lower portion of the distal end 406 of the shell 402 includes a molded groove 416 (e.g., slot, channel, valley, etc.) that a head or distal portion (e.g., brush) of the sample component 408 can sit or reside in to collect a sample as the shell 402 is moved or rotated in a radial direction by the user's finger. The groove 416 allows the distal end (e.g., head or brush portion) of the sample component 408 to reside in a specific location at the distal end of the shell 402. Such a location is near or proximate a distal tip of the user's finger when inserted into the shell 402 (FIGS. 6A and 6B). This can provide greater tactile contact or feedback between the user's finger, the sample component 408, and patient cavity for more accurate sample collection. For example, the groove 416 enables the user's finger to press on the distal end of the sample component 408 and ensure contact between the wall of a patient cavity and the sample component 408. The groove 416 can also prevent the distal end of the sample component 408 from shifting or moving side to side when residing in the groove 416.

The channel 410 also provides a space where the sample component 408 can be protected from contamination (e.g., mammalian tissue) prior to the sample component 408 being used to collect a sample at a desired sample site as described in more detail below. In some embodiments, the channel 410 may be used to insert other types of components to the sample collection site or otherwise into the patient. For example, a pretreatment component such as a foam brush can be inserted therethrough. In other embodiments, the channel 410 can be used to insert a vacuum tube in order to clean out the collection site or area (e.g., a cavity). Once the area is clean, the vacuum tube may be retracted and the sample component 408 can be inserted to collect a sample. Any components inserted into the proximal opening 414 will be positioned outside of the patient cavity. The component 408 can then be inserted through the channel 410 (e.g., beginning at a proximal opening 411) until a distal end of the component 408 exits through the distal opening 412 which will be inside the patient cavity.

In some embodiments, the distal opening 412 is protected and/or covered by a movable flap or cover. The flap or cover can prevent contaminants from entering the interior of the shell 402 through the opening 412 as the device 400 is inserted through the patient cavity. The flap or cover can be pushed or moved open by the sample component 408 or other components as described above when inserted through the opening 412. In other embodiments, a distal end 406 of the device 400 can include a stopper component 413. The stopper component 413 can prevent a component (e.g., the sample component 408) from extending past the distal tip or end 406 of the device 400. The stopper component 413 can also ensure that contact between the finger, device 400, and inserted component (e.g., the head or distal portion of the sample component 408) is maintained throughout the collection process.

In certain embodiments, as illustrated in FIGS. 4-7C, the device 400 can include a tab 420 (e.g., a projection portion) on the top or upper portion of the device 400 at the proximal end 404 to assist or help the user place the device 400 on a finger. The tab 420 may have one or more ridges 422 to provide increased gripping portions on the tab 420.

The device 400 can include curved or rounded edges to decrease resistance during insertion and increase maneuverability. In some embodiments, the distal end 406 of the device 400 may be designed with extruded geometry to help with removing debris during insertion. The device 400 can be composed of a flexible biocompatible material which may be stretched over the user's finger when being worn or mounted.

The device 400 can include one or more openings in the walls or surfaces of the device 400 (e.g., around the top or upper portion of the device 400) to minimize the surface area contact between the device 400 and the finger. This can simplify placing the device 400 on the finger. In certain embodiments, the device 400 can include an additional activation component 424 (e.g., a thumb rest) for activating the sample component 408 for collection once the finger worn device 400 is inserted into a patient cavity. The activation component 424 can be press fit or otherwise suitably secured onto the sample component 408. In some embodiments, the activation component 424 can be molded onto the sample component 408. For example, the activation component 424 can include a projection or tab at the proximal end of the sample component 408 for providing an additional traction or contact surface to activate the sample component 408 for collection.

In some embodiments, the sample collection device 400 can have a molded feature at the distal end to enable the sample component 408 to slide into a proper or desired location along the device 400 and/or in the patient cavity. The feature can also stop or prevent the sample component 408 from further or deeper insertion.

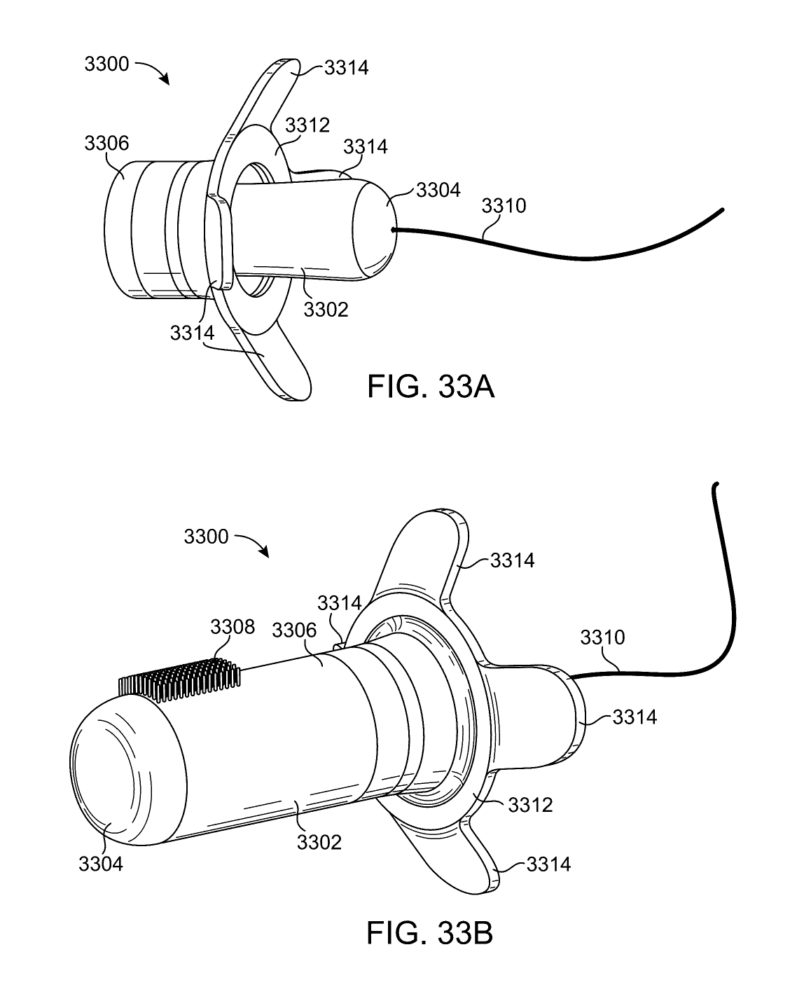

FIGS. 33A-B illustrate various side views of a flexible finger worn sample collection device 3300 (e.g., push in adaptor) configured in accordance with another embodiment of the present technology. The sample collection device 3300 is configured to be worn or placed onto a finger (e.g., the index finger) of a human subject. Referring to FIGS. 33A-B together, the sample collection device 3300 includes a flexible sample component 3302, having an inside surface (shown in FIG. 33A) and an outer surface (shown in FIG. 33B) shaped and sized to conform to a human subject's finger. The sample component 3302 includes a proximal end 3306 and a distal end 3304. The distal end 3304 of the sample component 3302 comprises at least one abrasive portion 3308. The sample component 3302 is configured to receive the user's finger at an inside surface of the distal end 3304. The sample collection device 3300 has a removal component 3310 (e.g., a string) securably attached to an inside surface of the distal end 3304 of the sample component 3302.

In certain embodiments, as illustrated in FIGS. 33A-B, the sample collection device 3300 can include a positioning component 3312 at the proximal end 3306 of the sample component 3302. The positioning component 3312 can contain one or more tabs 3314 (e.g., a projection portion) to assist or help the user place the device 3300 in a cavity of a patient as well as to manipulate the device to collect a sample. Both the sample component 3302 and positioning component 3312 can be formed from suitable flexible or rigid materials. In a preferred embodiment, the sample component 3302 is formed from a suitable flexible material such that the subject's finger can push against the inside surface of the distal end 3304 of the sample component 3302 to force the sample component 3302 extends into a cavity of a patient and to configure the abrasive head portion 3308 to be on an exposed surface of the sample component 3302. The abrasive head portion 3308 can be made of any suitable material and geometry.

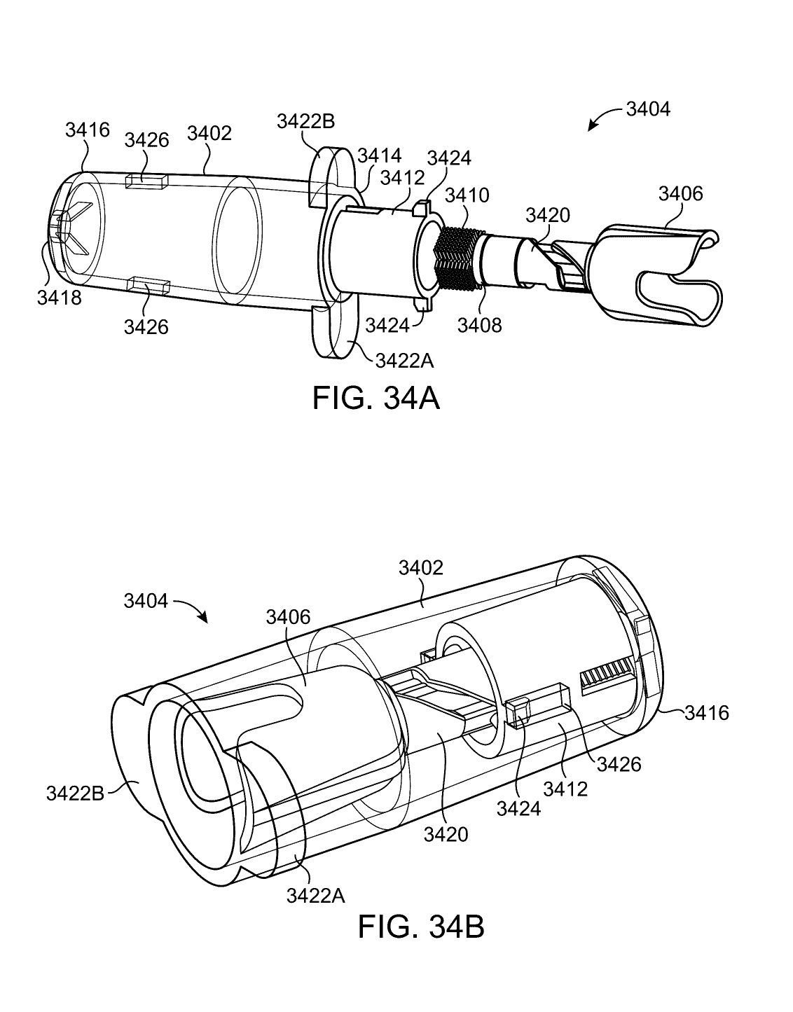

FIGS. 34A-B illustrate various side views of a finger worn sample collection device 3400 (e.g., push-and-twist) configured in accordance with an alternative embodiment of the present technology. The sample collection device 3400 is configured to be worn or placed onto a finger (e.g., the index finger) of a human subject. Referring to FIGS. 34A-B together, the sample collection device 3400 includes a proximal end and a distal end, for guiding the abrasive head portion 3410 into and through a cavity (e.g., an orifice or lumen) of a patient. The outer shield assembly 3402 includes a distal end 3416 opposite a proximal end 3414, wherein the distal end 3416 may comprise a flexible tip portion 3418. The proximal end 3416 of the outer shield assembly 3402 is configured for receiving a distal end 3408 of the inner component 3404 to be inserted therethrough. The inner component 3404 comprises a distal end 3406, a proximal end 3408, with a lumen extending therebetween. The proximal end 3408 of the inner component 3404 is configured to receive a finger of a human subject. The distal end 3408 of the inner component 3404 comprises an abrasive head portion 3410. The adapter 3412 is configured to be inserted into the lumen of the outer shield assembly at its proximal end 3414. The adapter 3412 is further configured to guide the abrasive head portion 3410 of the inner component 3404 to protrude past the distal end 3416 of the outer shield assembly 3402 thereby penetrating the flexible tip portion 3418. In some embodiments, the inner component has a twisting mechanism 3420 that is configured to allow the inner component to be released and/or locked. In some embodiments, by twisting the inner component 3404 within the adapter 3412, the inner component 3404 moves through the outer shield assembly and the abrasive head portion 3410 protrudes past the flexible tip portion 3418. The adapter 3412 can be formed from a suitable rigid material, semi-rigid material, and/or semi-flexible material. In some embodiments, the adapter 3412 is positioned in the outer shield assembly 3402 and held in place by cutouts in the outer shield assembly 3402 or extrusions 3424 in the adapter 3412 and extrusions 3426 in the outer shield assembly 3402. The extrusions 3424 in the adapter 3412 can be positioned symmetrically to allow the twisting mechanism 3420 to move. The twisting mechanism 3420 can also have extrusions that securably attach to the extrusions 3424 of the adapter 3412 which allows the abrasive head portion 3410 of the inner component 3404 to protrude past the distal end 3416 of the outer shield assembly 3402 and to lock the inner component in place during use.

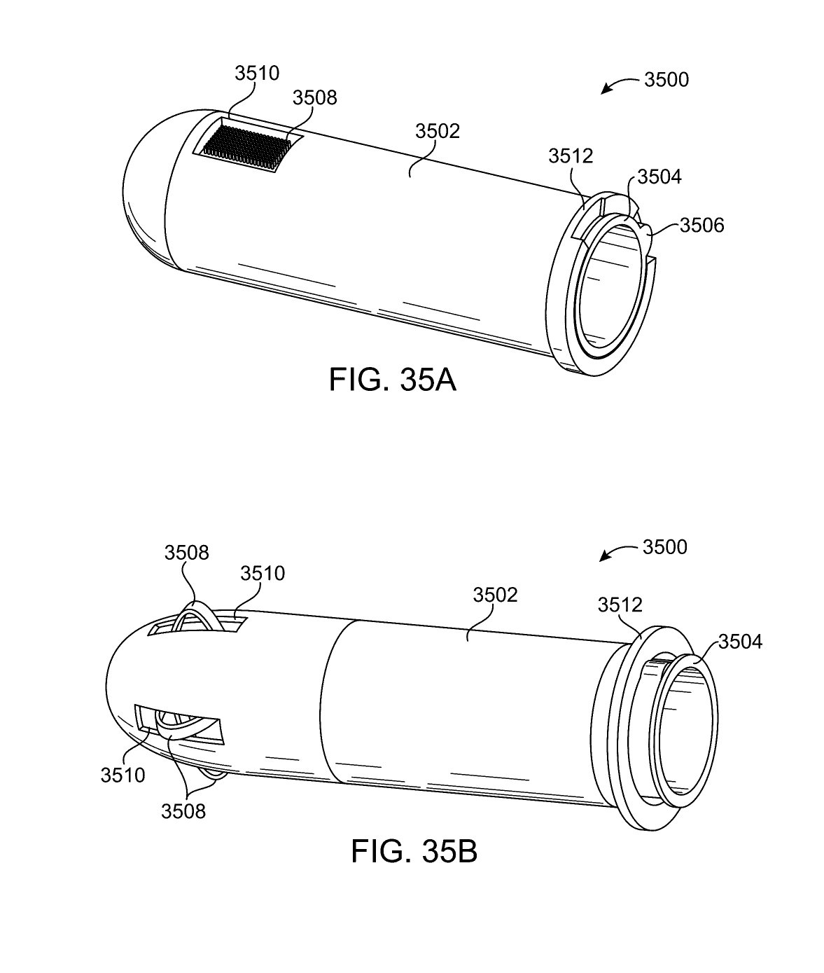

FIGS. 35A-B illustrate a finger worn sample collection device 3500 (e.g., push-and-twist and push-to-expand) configured in accordance with alternative embodiments of the present technology. The sample collection device 3500 is configured to be worn or placed onto a finger (e.g., the index finger) of a human subject. Referring to FIGS. 35A-B together, the sample collection device 3500 includes an outer shield assembly 3502 having a proximal end and a distal end and an inner component 3504 having a distal end and a proximal end. The outer shield assembly 3502 can be flexible or rigid. The inner component 3504 can be flexible or rigid. In some embodiments, the inner component 3504 is rotatable. The distal end of the inner component 3504 comprises at least one abrasive head portion 3508 and the proximal end is configured to receive a finger (e.g., index finger) of a human subject. The outer shield assembly 3502 is configured to have at least one aperture 3510. The abrasive head portion(s) 3508 protrudes past the at least one aperture 3510 when the inner component 3504 is twisted inside the outer shield component 3502 (as shown in FIG. 35A) or when the inner component is pushed through the outer shield component 3502 (as shown in FIG. 35B). In some embodiments, the outer shield assembly 3502 can be configured to have a covering over the aperture 3510 that can be opened to expose the abrasive head portion 3508 when needed. In some embodiments, the sample collection device 3500 can also comprise a locking mechanism to secure the inner component 3504 in the outer shield assembly 3502. The locking mechanism can be positioned anywhere on the sample collection device 3500, but in a preferred embodiment it is positioned at or near the proximal end of the outer shield assembly 3502. When the inner component 3504 is twisted it can lock into position with the outer shield assembly 3502. In other embodiments, the inner component 3504 locks into position with the outer shield assembly 3502 when the inner component 3504 is pressed into the outer shield assembly 3502. In another embodiment, the sample collection device 3500 includes a release mechanism configured to protrude the abrasive head 3508 past the aperture 3510 of the outer shield assembly 3502. The abrasive head 3508 can be bristles, foam (e.g., expandable), abrasive polymer, or adhesive, plastic, silicone, or other suitable materials. In some embodiments, the abrasive head 3508 is flexible and/or bendable such that by compressing the ends it may bend and protrude through the aperture 3510 of the outer shield assembly 3502.

A protective barrier 3512 can be secured or coupled to a distal end of the outer shield assembly 3500. In some embodiments, the inner component 3504 can be configured to have a tab 3506 on the proximal end of the inner component 3504 which functions to lock or releasably secure the inner component 3504 into the outer shield assembly 3502 when the at least one abrasive head portion 3508 aligns with the 3510 at least one aperture.

Method of Collection with Sample Collection Device

An example method of collecting a sample (e.g., a tissue sample) with the sample collection device 400 can include one or more of the following steps in accordance with an embodiment of the present technology. The sample collection device 400 can be removed from a packaging or other wrapping that it is stored in. The sample collection device 400 can be pre-lubricated or a physician or other user can lubricate an outer surface of the device 400 before inserting the device 400 into the patient. The device 400 is then placed on the user's finger 418 and inserted into the patient cavity. Once the device 400 is located at the desired tissue sample site, the sample component 408 is inserted through proximal opening 414 of the device 400 and/or the channel 410. The sample component 408 is inserted until it exits the distal opening 412 and reaches the distal end 406 of the device 400 and/or finger. In some embodiments, as described above, the sample component 408 can be inserted past the distal end 406 of the device 400.

For collection, the user moves its finger 418 in a radial or circular motion to collect a sample (e.g., a tissue sample). Once the sample is collected, the sample component 408 can be removed from device 400. After the sample component 408 is removed, the device 400 can be removed from the cavity and the user's finger and discarded. In some embodiments, the device 400 can be decontaminated and reused for another sample collection.

An example method of collecting a sample (e.g., a tissue sample) with the sample collection device 3300 can include one or more of the following steps in accordance with an embodiment of the present technology. The sample collection device 3300 can be removed from a packaging or other wrapping that it is stored in. The sample collection device 3300 can be pre-lubricated or a physician or other user can lubricate an outer surface of the device 3300 before inserting the device 3300 into the patient. The device 3300 is then positioned into or near a patient cavity with the flexible sample component 3302 configured away from the patient cavity such that the abrasive head portion is not exposed, as depicted in FIG. 33A. The positioning component 3312 can be held by the user's hand and may be used to twist or otherwise configure the device 3300 into position. Once the device 3300 is located at the desired tissue sample site, the sample component 3302 is inserted into the patient cavity by the user pushing the inside surface of the distal end 3304 of the sample component 3302 into the patient cavity, as depicted in FIG. 33B. This exposes the abrasive head portion 3308 to the sample site.

For collection, the user moves its finger in a radial or circular motion to collect a sample (e.g., a tissue sample). Once the sample is collected, the user holds that device 3300 in position with one hand on the positioning component 3312 and/or tabs 3314 and pulls the removal component 3310 with its other hand to retract the abrasive head portion 3308 so that the abrasive head portion is not exposed, as in FIG. 33A. After the abrasive head portion is retracted within the sample component 3302, the device 3300 can be removed from the cavity. In some embodiments, the device 3300 can be decontaminated and reused for another sample collection.

Another example method of collecting a sample (e.g., a tissue sample) with the sample collection device 3400 can include one or more of the following steps in accordance with an embodiment of the present technology. The sample collection device 3400 can be removed from a packaging or other wrapping that it is stored in. The sample collection device 3400 can be pre-lubricated or a physician or other user can lubricate an outer surface of the device 3400 before inserting the device 3400 into the patient. The device 3400 is then placed on the user's finger and inserted into the patient cavity. Once the device 3400 is located at the desired tissue sample site, the inner component 3404 is inserted through the lumen of the outer shield assembly 3402 until it engages with the adapter 3412. The protrusions 3424 on the adapter 3412 can guide the abrasive head portion 3410 of the inner component 3404 to protrude past the distal end 3416 of the outer shield assembly 3402 thereby penetrating the flexible tip portion 3418.

Once the sample is collected, the inner component 3404 can be withdrawn back into the outer shield assembly 3402 and the sample collection device 3400 can be removed from the cavity and the user's finger and the outer shield assembly discarded. In some embodiments, the device 3400 can be decontaminated and reused for another sample collection.

An example method of collecting a sample (e.g., a tissue sample) with the sample collection device 3500 can include one or more of the following steps in accordance with an embodiment of the present technology. The sample collection device 3500 can be removed from a packaging or other wrapping that it is stored in. The sample collection device 3500 can be pre-lubricated or a physician or other user can lubricate an outer surface of the device 3500 before inserting the device 3500 into the patient. The device 3500 is then placed on the user's finger and inserted into the patient cavity. Once the device 3500 is located at the desired tissue sample site, the inner component 3504 can be twisted to expose the abrasive head portion 3508 past the aperture 3510 of the outer shield component 3502. The user may move its finger in a radial or circular motion to collect a sample (e.g., a tissue sample). Once the sample is collected, the inner component 3504 may be rotated to retract the abrasive head 3508 back through the aperture 3510. The device 3500 can then be removed from the cavity and the user's finger and discarded. In some embodiments, the device 3500 can be decontaminated and reused for another sample collection.

### Abrasive Sample Component

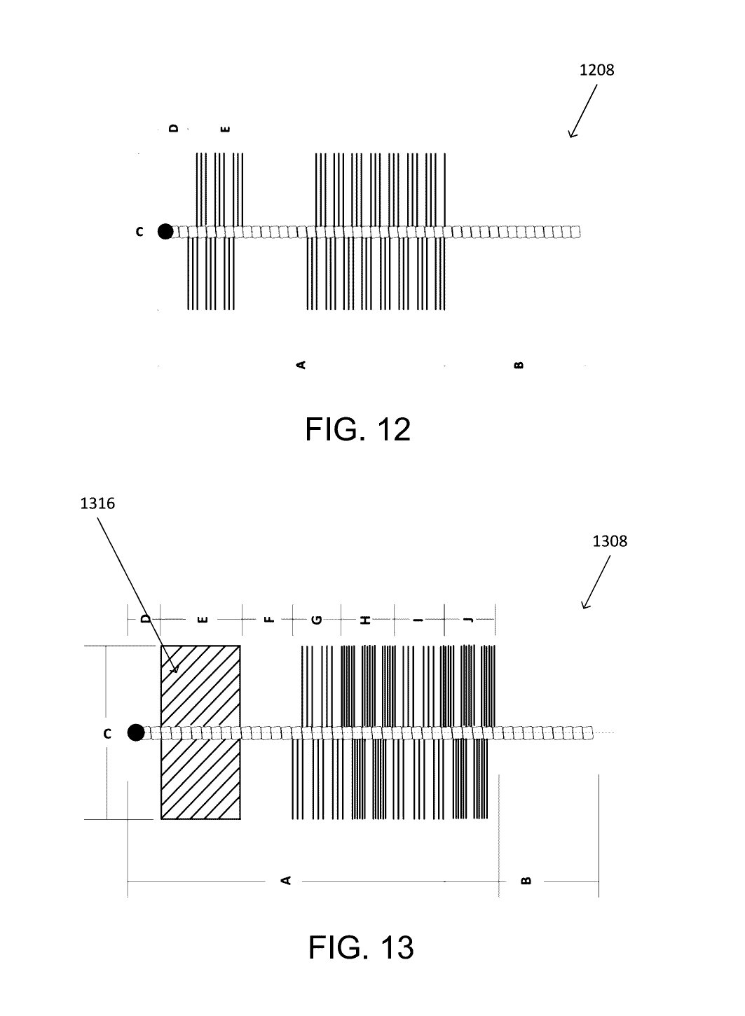

FIGS. 8-21B illustrate side views of various sample components 808, 908, 1008, 1108, etc. (e.g., a cytobrush, brush, abrasive head, etc.) configured in accordance with embodiments of the present technology that can be used with the sample collection devices (e.g., 100, 400, etc.) as described herein or other types of sample collection devices. The sample components 808, 908, etc. can include an abrasive head, distal, or end portion 810 (e.g., one or more bristle sections as described in more detail below) for collecting samples. For example, referring to FIG. 8, the sample components 808, etc. can include a flexible metal wire 812 with strands twisted together. The flexible twisted metal wire 812 can support the multiple bristles 814 positioned at the head portion 810. The bristles 814 can have any suitable shape, size, rigidity, and density. Diameters (e.g., radial distance from the wire 812) of the bristles 814 along the axis of the wire 812 may be generally constant (as illustrated in FIG. 8) or may have varying diameters (FIGS. 9 and 10).

The bristles 814 can contact the lumen walls of a patient cavity or lumen (e.g., a rectum) to collect a sample. The bristle density may vary along the length of the wire as can the bristle thickness. The diameter and shape of the bristles 814 can be suitably sized and shaped to ensure sufficient contact with the lumen walls for accurate sample collection. During collection, the force exerted by the bristles 814 must be sufficient to ensure abrasion against the lumen walls. Such force can be varied or changed by using a variety of bristle types (e.g., hardness, stiffness, rigidity, diameter), varying geometry or shape, and/or changing flexibility of the twisted wire 812.

Because the shape of a patient cavity (e.g., orifice or lumen) can vary or be irregular, the sample component 808, 908, etc. is flexible enough to bend and make contact with the lumen walls during twisting of the brush and/or movement in the radial and back and forth directions. In some embodiments, a distal end of the sample component 808, 908, etc. may require a smaller diameter bristle 814 length to ease insertion, gently open the lumen, fit into a nook, and/or for brushing to collect a sample (FIG. 9). However, some collection protocols may require the distal end of the bristles 814 sample component having a greater diameter as compared to the proximal end (FIG. 10). In certain embodiments, as illustrated in FIG. 9, the proximal and distal ends of the bristles 814 can have generally equal diameters that increase in size toward a middle portion of the abrasive head portion 810

During insertion, the distal end of the head portion 810 (e.g., bristles 814) will be inserted through the sample devices and initially contact the lumen walls and abrade the surface of the lumen walls. By having smaller diameter bristles 814 at the distal end, the contact with the inner walls of the sample device as the sample component is inserted therethrough is minimized or reduced. This can minimize or reduce any cross contamination between the bristles 814 and device 100, 400, etc.

As illustrated in the various embodiments, the bristles 814 may be arranged in multiple configurations such that there can be sections with varying bristle density (FIGS. 8-15). For example, individual bristles 814 or bristle sections can be spaced apart at varying gaps and distances (FIGS. 11 and 12). In some embodiments, the bristle sections (e.g., abrasive portions) include more or less bristles 814 along the length of the wire 812. Further, the bristle sections can be positioned at varying locations along the length of the wire 812 (e.g., more proximate the distal end, more proximate the proximal end, etc.). The bristles 814 or bristle sections can have varying sizes and stiffnesses in sections along the length of the wire as illustrated by the varying distances between the bristles 814 and line weights of the bristles 814 (FIG. 13).

In certain embodiments, additional materials 1316 can be placed or coupled onto the front or distal end 810 of the sample components. The additional materials can be used to clean or prepare the collection area, protect the channel from contamination, move contaminants or take in contamination (FIGS. 13-16). The additional material may be plastic, foam, rubber, metal or other alternative materials that are biocompatible and safe. The material may be expandable, such as foam. As the sample component is inserted, the material can expand inside the cavity and/or sample collection device (FIG. 17). The expandable material may be used to clean the area or device before collection, make room in the cavity, and take in fluid so that the fluid does not reach the brush or flow through the channel (e.g., of the cavity and/or sample collection device).

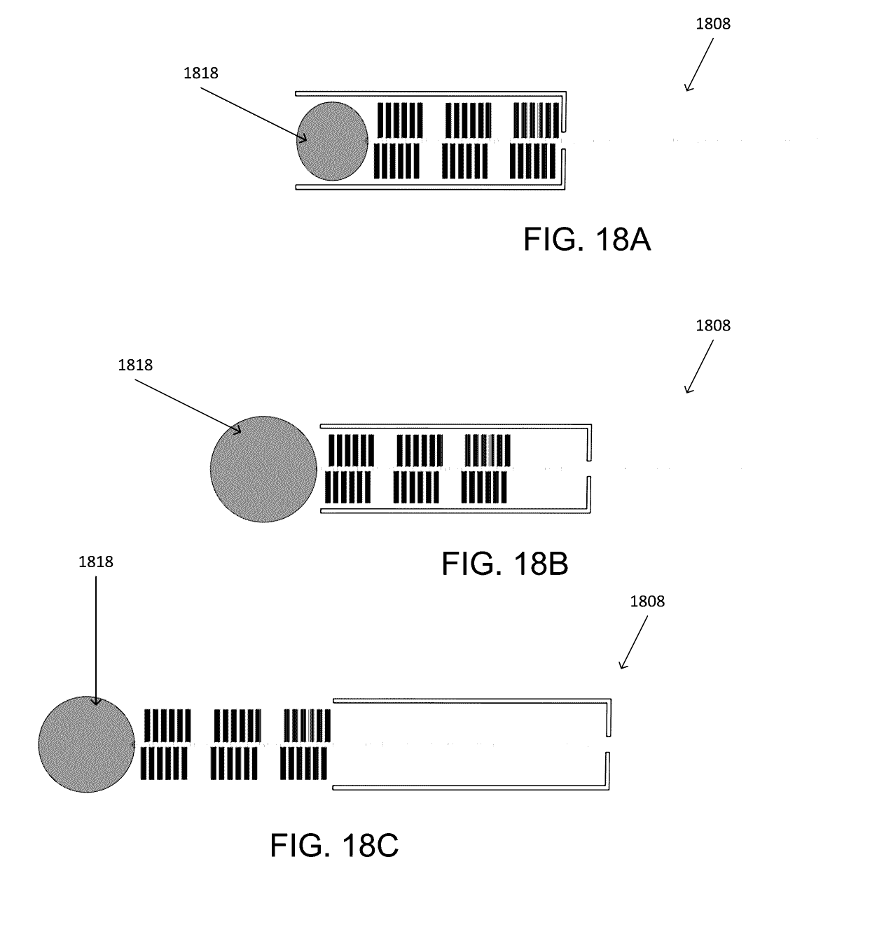

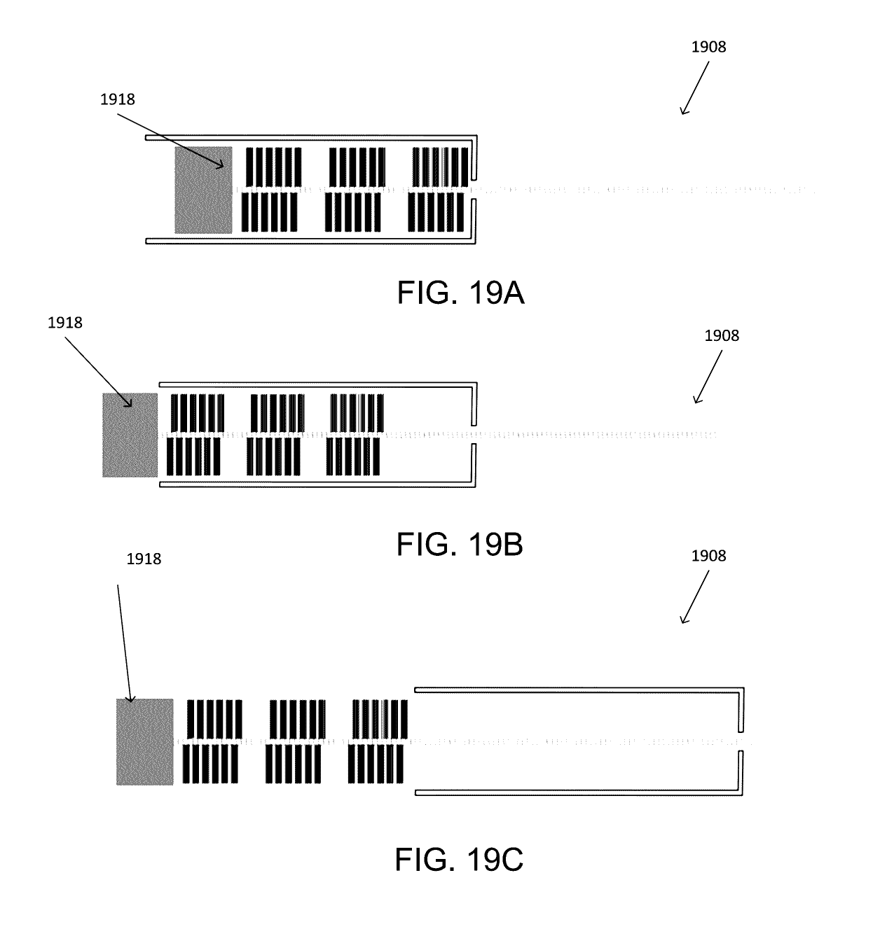

In some embodiments, sample component 808, 908, etc. can have a generally thin protective cover 1718 that protects the bristles 814, the tip portion 810, and/or any other material along the length of the sample component (FIG. 17). The protective cover 1718 can move (e.g., expand and retract) as the sample component is inserted and retracted to help expose and cover the sample component (FIGS. 18A-18C and 19A-19C). In some embodiments, multiple protective barriers or covers can be used to isolate the materials on the distal front or tip of the sample component 808, 908, etc. from the collection bristles 814 (FIGS. 18A-18C and 19A-19C).

The distal tip of the sample component 808, 908, etc. can be blunt or round as illustrated in the various embodiments (FIG. 8). The distal tip may be any suitable size (FIGS. 8, 13 and 15). The blunt distal tip can serve multiple purposes. For example, it can prevent or reduce patient discomfort during insertion of the sample component. Additionally, the greater the bluntness, the greater the surface area, and the greater distribution of force during contact between the sample component and the tissue. This can ensure generally quick, easy and/or painless insertion. Minimizing any rigid and sharp edges can also diminish the potential of puncturing tissue.

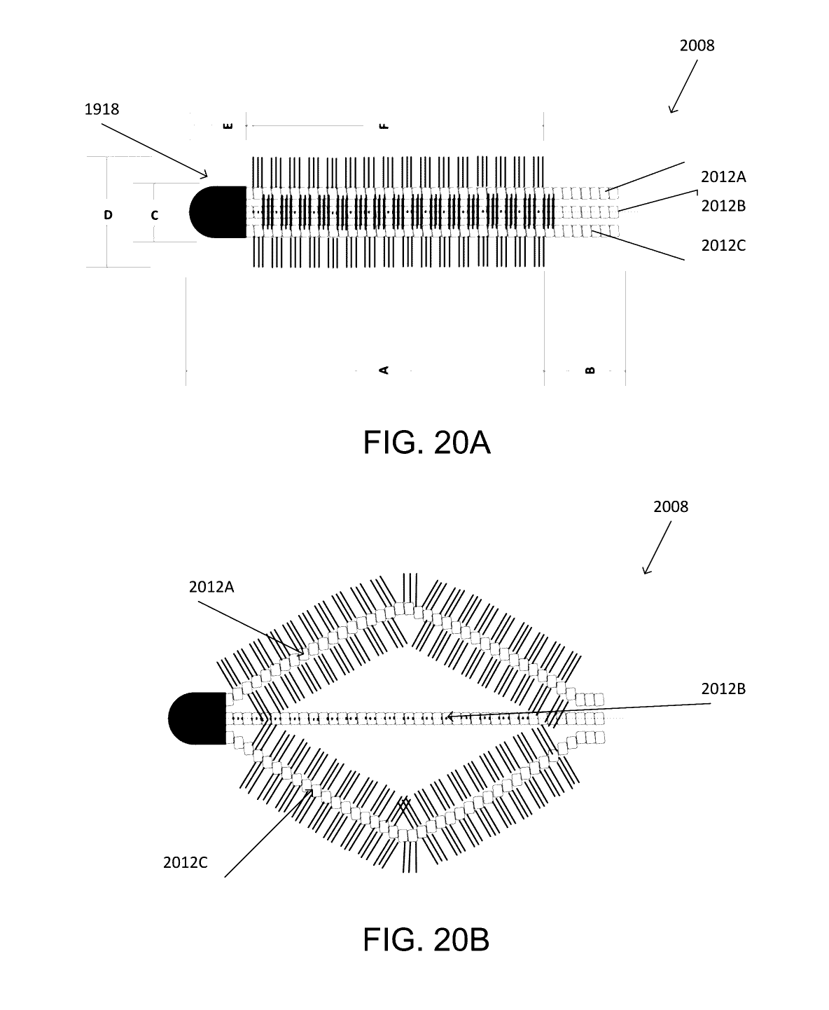

The sample components can have multiple independent abrasive surfaces. For example, as illustrated in FIGS. 20A-20B, the sample component 2008 can include one or more flexible wires 2012 (e.g., identified individually as a first wire 2012A, second wire 2012B, and third wire 2012C). All the wires 2012A-2012C can include one or more abrasive surfaces (e.g., multiple bristle portions or sections). In some embodiments, only certain wires include abrasive surfaces (e.g., wires 2012A and 2012C). As illustrated, the wires 2012A and 2012C can include multiple abrasive surfaces which are contracted inside a sample collection device (FIG. 20A) and expand and spring out (FIG. 20B) once inserted into, for example, a mammalian cavity.

In some embodiments, The sample component 808, 908, etc. can include a balloon 2120 with multiple extruded fingers 2122 on the outside of the balloon 2120. Once inserted past the distal end of a sample collection device, the balloon 2120 can be inflated. This allows the fingers 2122 to make contact with the lumen walls, whereby when twisted, will abrade the surface (FIGS. 21A-21B) to collect a sample. The abrasive balloon 2120 will expand to achieve maximum contact with the lumen walls.

### Glove-Based Abrasive Sample Collection Device

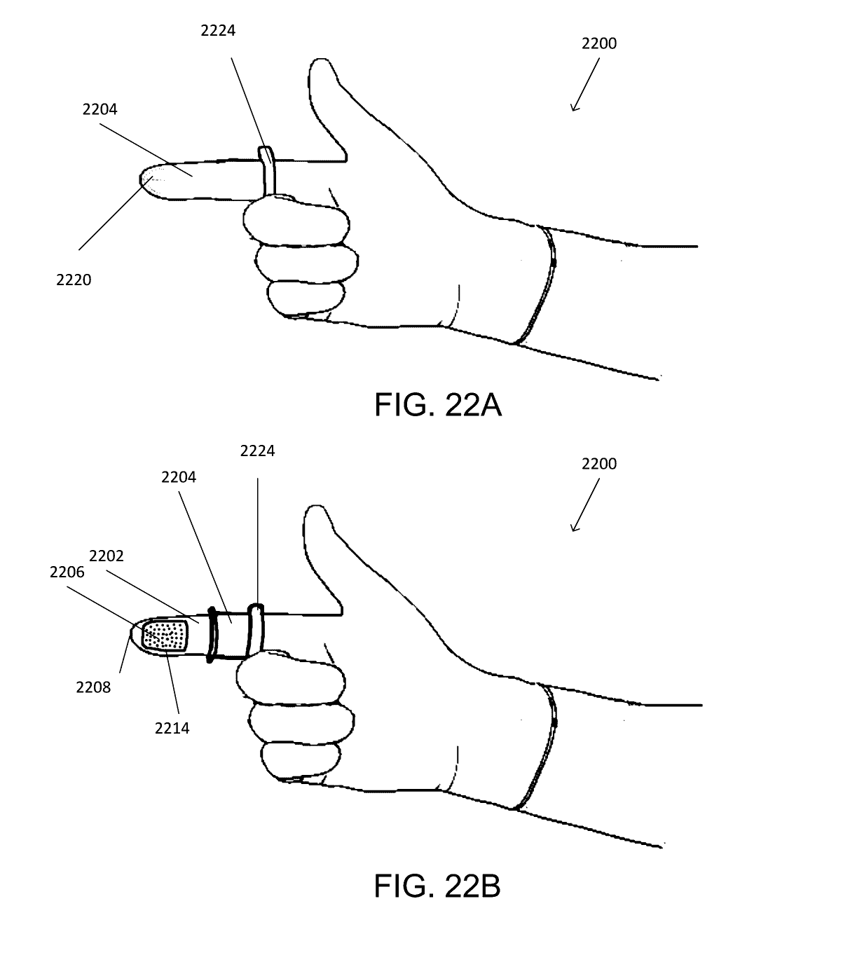

FIGS. 22A-24 illustrate various side views and bottom views of a glove-based sample collection device 2200 configured in accordance with another embodiment of the present technology. Referring to FIGS. 22A-24 together, the sample collection device 2200 is configured to be worn over the hand of a user (e.g., a glove). In some embodiments, the sample collection device 2200 can be worn on only a portion of the hand of a user (e.g., one or more fingers). The sample collection device 2200 includes an inner layer 2202 (e.g., liner) that is protected (e.g., covered or shielded) by an outer layer 2204 (e.g., liner). The inner layer 2202 can include an abrasive pad 2206 (e.g., an abrasive material, portion, or section) positioned on a bottom of a distal tip of a finger portion 2208 of the inner layer 2202 for collecting a sample. The abrasive pad 2206 can be composed of a variety of materials. For example, the abrasive pad 2206 can be composed of silicone. The abrasive pad 2206 can include multiple bristles 2214. The height, density, spacing, stiffness, rigidity, and/or diameter of the bristles 2214 may vary. The bristles 2214 may be made from plastic, silicone, or other suitable materials.

In some embodiments, the inner layer 2202 can include another material on the distal tip of the finger portion 2208. For example, the inner layer 2202 can be overmolded with a silicone feature or have a silicone feature secured onto the layer with an adhesive. This silicone feature may have ribs, bristles, or other extrusions. One alternative to silicone would be a plastic component which may be fixed to the inner layer 2202 with adhesive. This layer may enable material from being moved away to provide access to a surface (for example, the rectal lining).

The outer layer 2204 is configured to protect and/or cover the abrasive pad 2206 during insertion and retraction of the collection device 2200 into and out of the patient cavity. The collection device 2200 can include a stopper component 2224 (e.g., over-molded ring element) positioned (e.g., extending radially about) on the outer layer 2204 to prevent the collection device 2200 from being inserted farther into patient cavity. In some embodiments, the outer layer 2204 can include multiple layers. For example, the outer layer 2204 can include two or more layers to increase thickness, while still providing flexibility. In other embodiments, the distal tip of the outer layer 2204 may also have an additional layer of material to enable clearing of the sample collection area of fluid or other debris.

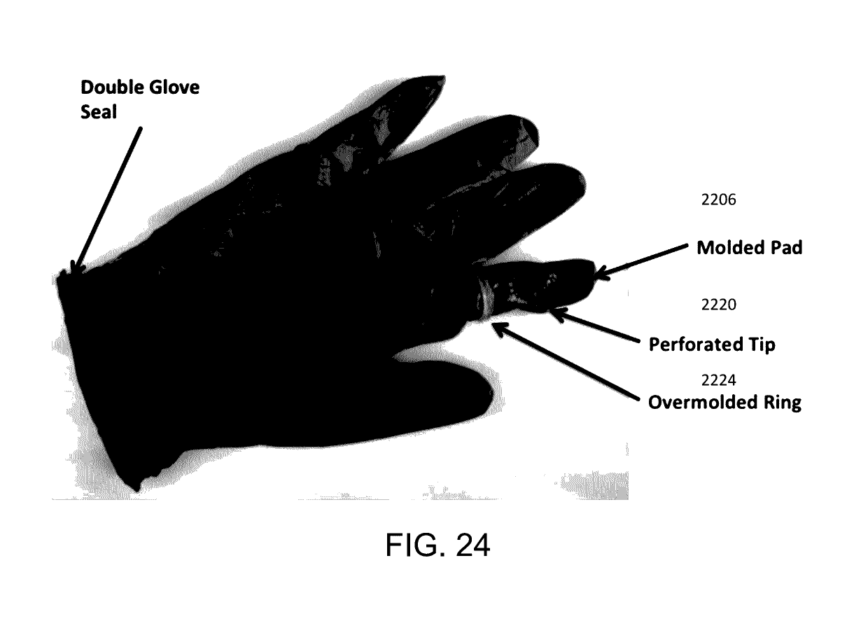

In operation, the distal tip or end of the outer layer 2204 distal tip will open during insertion exposing the abrasive pad 2206 (FIG. 22B) underneath the outer layer 2204. For example, the outer layer distal tip can be perforated 2220 (e.g., include one or more flaps) configured to open (e.g., spread outwardly or roll away from the tip) once the device 2200 is inserted (FIGS. 22B and 24). The outer layer 2204 distal tip may be cut to allow the inner layer 2202 to be exposed to the cavity or lumen wall (FIG. 24). The outer layer 2202 may be removed, folded, rolled or moved by pulling an additional piece (e.g., string, tab, etc.) to expose the inner layer 2202 or abrasive pad 2206. In some embodiments, the outer layer 2204 may be opened or released by pushing on another piece or portion which is attached to the distal end of the device 2200 (e.g., the abrasive pad 2206 or stopper component 2224). For example, in some embodiments, a molded lever can extend between the distal end to some area proximal to the distal end. When the device 2200 is inserted into a cavity, once the outer area of the cavity makes contact with the lever arm, it opens the distal end (e.g., of the outer layer 2204). As the distal end opens, the inner layer 2202 may be exposed and is inserted into the cavity.

In some embodiments, the inner layer 2202 may be inserted through a side opening in the outer layer 2204. The outer layer 2204 may have a slit on either side which may be collapsed (e.g., resiliently deforms) when nothing is being forced through. However, if the inner layer 2202, which will have a finger inside, slides through the slit, the outer layer 2204 can expand and open, allowing said inner layer 2202 to extend through the outer layer 2204. The abrasive pad 2206 may then contact the collection area or site to collect a sample.

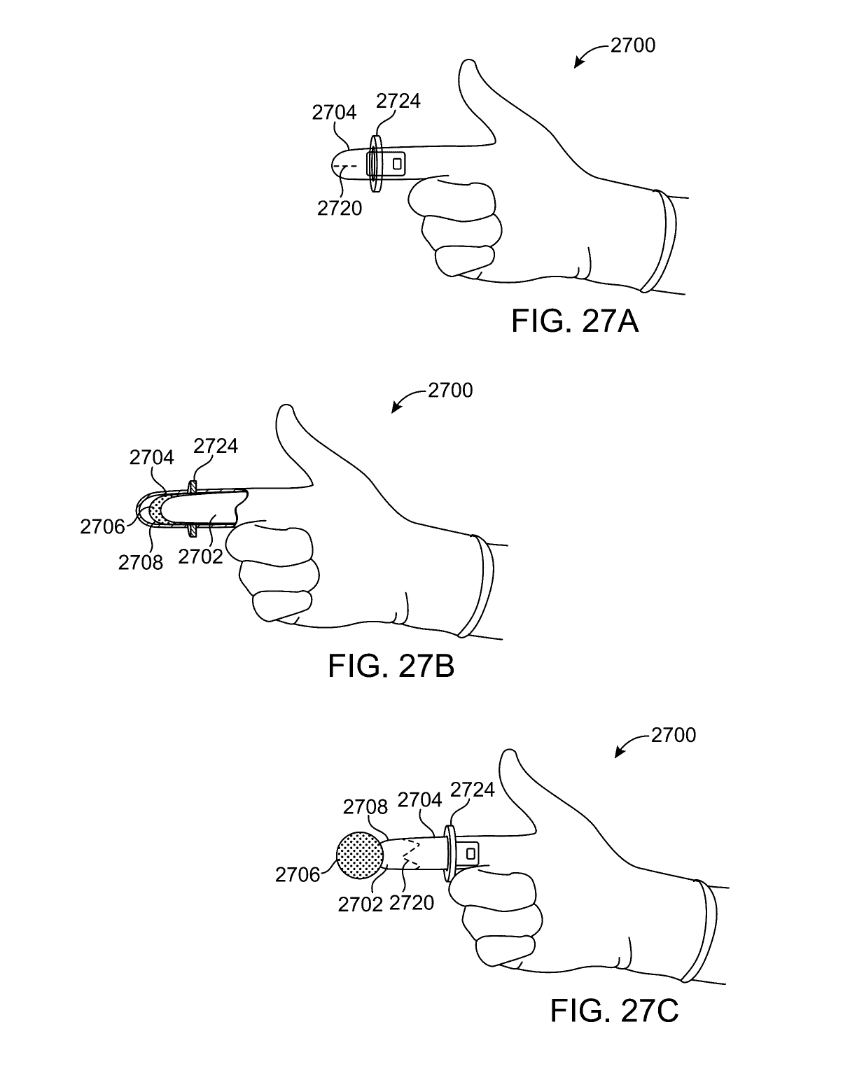

FIG. 27 illustrates various side views of a glove-based sample collection device 2700 configured in accordance with another embodiment of the present technology. The sample collection device 2700 is configured to be worn over the hand of a user (e.g., a glove). In some embodiments, the sample collection device 2700 can be worn on only a portion of the hand of a user (e.g., one or more fingers). The sample collection device 2700 includes an inner layer 2702 (e.g., liner) that is protected (e.g., covered or shielded) by an outer layer 2704 (e.g., liner). The inner layer 2702 can include a sample component 2706 (e.g., an abrasive material, expandable foam, etc.) positioned on a distal tip of a finger portion 2708 of the inner layer 2702 for collecting a sample. The sample component 2706 can be composed of a variety of materials. For example, the sample component 2706 can be composed of an expandable foam, plastic, silicone, or other suitable materials (shown in FIG. 27C in expanded an configuration).

In some embodiments, the inner layer 2702 can include another material on the distal tip of the finger portion 2708. For example, the inner layer 2702 can be overmolded with a silicone feature or have a silicone feature secured onto the layer with an adhesive. This silicone feature may have ribs, bristles, or other extrusions. One alternative to silicone would be a plastic component which may be fixed to the inner layer 2702 with adhesive. This layer may enable material from being moved away to provide access to a surface (for example, the rectal lining).

The outer layer 2704 is configured to protect and/or cover the sample component 2706 during insertion and retraction of the collection device 2700 into and out of the patient cavity. The collection device 2700 can include a stopper component 2724 (e.g., over-molded ring element) positioned (e.g., extending radially about) on the outer layer 2704 to prevent the collection device 2700 from being inserted farther into patient cavity. In some embodiments, the outer layer 2704 can include multiple layers. For example, the outer layer 2704 can include two or more layers to increase thickness, while still providing flexibility. In other embodiments, the distal tip of the outer layer 2704 may also have an additional layer of material to enable clearing of the sample collection area of fluid or other debris. In operation, the distal tip or end of the outer layer 2704 distal tip will open during insertion exposing the sample component 2706 (FIG. 27C) underneath the outer layer 2704. For example, the outer layer distal tip can be perforated 2720 (e.g., include one or more flaps) configured to open (e.g., spread outwardly or roll away from the tip) once the device 2700 is inserted (FIGS. 27B-C). The outer layer 2702 may be removed, folded, rolled or moved by pulling an additional piece (e.g., string, tab, etc.) to expose the inner layer 2702 or sample component 2706. In some embodiments, the outer layer 2704 may be opened or released by pushing on another piece or portion which is attached to the distal end of the device 2700 (e.g., the sample portion 2706). For example, in some embodiments, a molded lever can extend between the distal end to some area proximal to the distal end. When the device 2700 is inserted into a cavity, once the outer area of the cavity makes contact with the lever arm, it opens the distal end (e.g., of the outer layer 2704). As the distal end opens, the inner layer 2702 may be exposed and is inserted into the cavity.

### Glove-Based Conduit Sample Collection Device

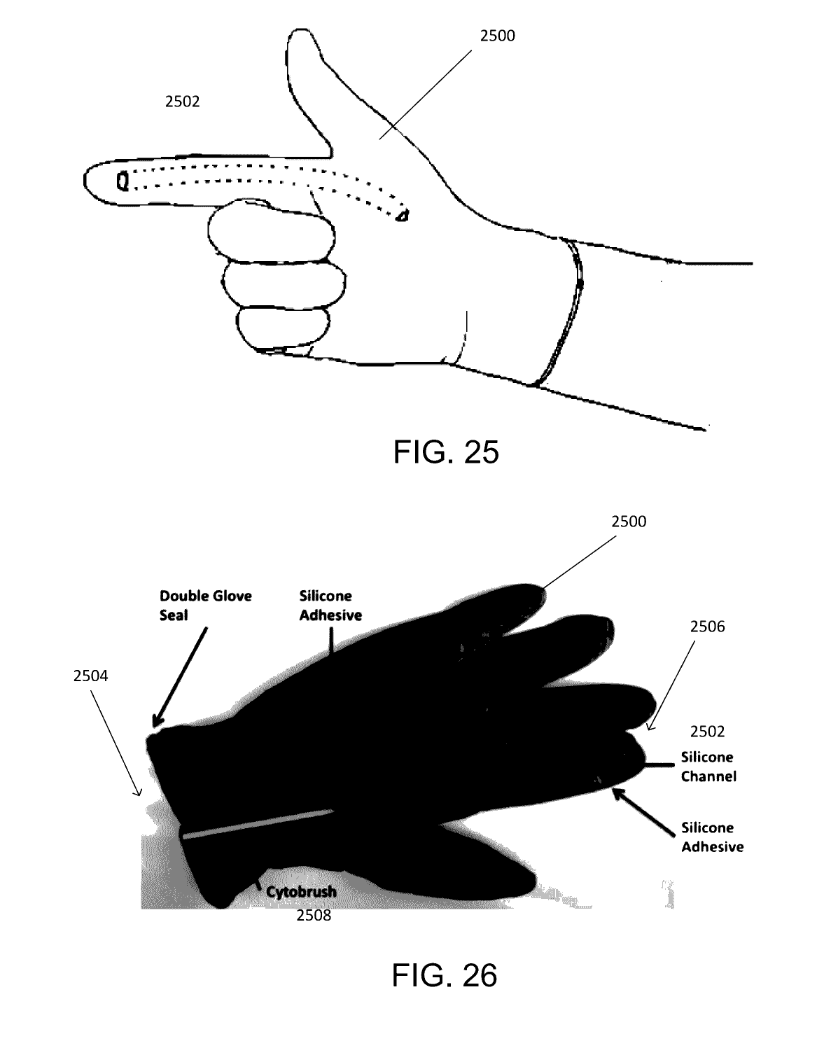

FIGS. 25 and 26 illustrate side and bottom views, respectively of a glove-based sample collection device 2500 configured in accordance with another embodiment of the present technology. Referring to FIGS. 25 and 26 together, the embodiment consists of a glove-based configuration having an outer layer in the shape of a hand that is generally similar to the configuration in FIGS. 22A-24. However, the embodiment of FIGS. 25 and 26 includes a channel 2502 (e.g., conduit, sheath, tube, lumen, etc.) that extends from the proximal end 2504 to the distal end 2506 (e.g., from the wrist portion to the distal end of a finger portion) of the sample collection device 2500 (FIG. 25). The channel 2502 is open on the proximal end or side (FIGS. 25 and 26) to receive a sample component therethrough as described in more detail below.

The distal end of the channel 2502 may be open. In some embodiments, the distal end of the channel 2502 may include a protective flap or cover to shield the channel 2502 from contaminants or debris as the device 2500 is inserted into a patient cavity. The channel 2502 is sized to support various components (e.g., a sample component, cytobrush, vacuum conduit, irrigation conduit, cleaner component, etc.) to be inserted through the channel 2502 and into the patient cavity.

The channel 2502 can be comprised of a flexible material. In other embodiments, the channel 2502 can be formed from a suitable rigid material, semi-rigid material, and/or semi-flexible material. The channel 2502 can be a separately formed component that is fixed or secured to the device 2500 by an adhesive or other suitable fastener. In other embodiments, the channel 2502 can be molded as one piece with the device 2500 or cut or formed through a layer of the device 2500.

The distal tip of the finger of the device 2500 has space for the sample component 2508 or other component to rest or reside at the tip whereby a user can provide a force on the sample component 2508. The distal tip of the device 2500 could have a molded end or stopping component to stop or prevent any component from being inserted beyond the distal tip.

From the foregoing, it will be appreciated that specific embodiments of the present technology have been described herein for purposes of illustration, but that various modifications may be made without deviating from the scope of the present technology. Specific elements of any of the foregoing embodiments can also be combined or substituted for elements in other embodiments. Furthermore, while advantages associated with certain embodiments of the present technology have been described in the context of these embodiments, other embodiments may also exhibit such advantages, and not all embodiments need necessarily exhibit such advantages to fall within the scope of the technology. Accordingly, the present technology is not limited, except as by the appended claims.

I/We claim:

1. A sample collection device, comprising:
an outer shield assembly comprising a distal end, a proximal end and a lumen extending therebetween, wherein the distal end comprises a flexible tip portion; and
an inner component comprising a distal end, a proximal end and a lumen extending therebetween, wherein the distal end of the inner component is configured to be inserted into the lumen of the outer shield assembly at its proximal end and to protrude past the distal end of the outer shield assembly thereby penetrating the flexible tip portion when the outer shield assembly and the inner component are in an assembled configuration.

2. The sample collection device of claim 1, further comprising a sample component having an abrasive head portion at a distal end, wherein the sample component is configured to be inserted into the lumen of the inner component at its proximal end and to protrude past the distal ends of the inner component and outer shield assembly when the outer shield assembly and the inner component are in an assembled configuration.

3. The sample collection device of claim 1, wherein the inner component comprises a stopper secured or attached to the proximal end for contacting the proximal end of the outer shield assembly and thereby limiting a distance the distal end of the inner component can protrude past the distal end of the outer shield assembly when the outer shield assembly and the inner component are in an assembled configuration.

4. The sample collection device of claim 1, wherein the flexible tip portion is configured to open when the distal end of the inner component protrudes past the distal end of the outer shield assembly.

5. The sample collection device of claim 1, wherein the flexible tip portion is unitarily or monolithically formed with the outer shield assembly.

6. The sample collection device of claim 1, wherein the flexible tip portion is rounded and outwardly spreadable relative to the distal end of the outer shield assembly.

7. The sample collection device of claim 1, further comprising a handle comprising a distal end and proximal end coupled the outer shield assembly.

8. The sample collection device of claim 7, further comprising a protective barrier secured or coupled to the distal end of the handle.

9. The sample collection device of claim 2, wherein the abrasive head portion comprises one or more bristles.

10. The sample collection device of claim 2, wherein the sample component is configured to be inserted into a cavity of a patient to a desired sample collection site in the patient through the lumen of the inner component.

11. The sample collection device of claim 2, wherein the sample component comprises a threaded portion at a proximal end.

12. The sample collection device of claim 11, wherein the threaded portion of the sample component is configured to securably attach to a threaded fitting.

13. The sample collection device of claim 11, wherein when the threaded portion of the sample component is securably attached to a threaded fitting, rotational force applied to the threaded fitting imparts a rotational motion on the sample component.

14. The sample collection device of claim 1, wherein the inner component is configured to be releasably secured to the outer shield assembly.

15. A sample component comprising a flexible member and an abrasive head having a plurality of abrasive surfaces comprising two or more wires wherein the wires comprise abrasive surfaces.

16. The sample component of claim 15, wherein at least one of the wires is a flexible wire.

17. The sample component of claim 15, wherein at least one of the wires are configured to expand once inserted into a patient cavity.

18. A method of collecting a biological sample using the sample collection device of claim 1 comprising:
inserting the outer shield assembly and the flexible tip portion into a patient cavity;
pushing the inner component through the lumen of the outer shield assembly to protrude the inner component past the distal end of the outer shield assembly thereby opening the flexible tip portion;
inserting a sample component through the lumen of the inner component and outer shield assembly until an abrasive head at a distal end of the sample component extends past the distal end of the inner component; and
collecting a biological sample from the patient cavity on the sample component.

19. The method of claim 18, wherein at least a portion of a sample collection device is pre-lubricated prior to insertion into a patient cavity.

20. The method of claim 18 further comprising imparting a rotational motion on the sample component and/or abrasive head to collect the biological sample from the patient cavity.
